(19) United States
(12) Patent Application Publication
(10) Pub. No.: US 2023/0055370 A1
(43) Pub. Date: Feb. 23, 2023

(54) METHODS FOR STAGING OF DISEASES

(71) Applicant: Healios GmbH, Basel (CH)

(72) Inventors: Guilhem DUPONT, Basel (CH); Markus EHRAT, Madgen (CH); John DUNNE, Dublin (IE)

(73) Assignee: Healios GmbH, Basel (CH)

(21) Appl. No.: 17/792,048

(22) PCT Filed: Jan. 8, 2021
(86) PCT No.: PCT/EP2021/050272

§ 371 Date: Jul. 11, 2022

(30) Foreign Application Priority Data
Jan. 9, 2020 (EP) 20151044.3

(51) Int. Cl. G16H 50/20; G16H 30/40; G01N 33/564

(52) U.S. Cl. CPC G16H 50/20; G16H 30/40; G01N 33/564; G01N 2800/285; G01N 2800/52; G16H 10/20

(57) ABSTRACT

The present invention relates to methods for providing the state of a disease in a subject comprising the steps of determining at least two parameters indicative of the state of a disease in the subject at a first time point; combining the determined parameters to provide a signature indicative of the disease state at the first time point; repeating the previous steps to provide at least one further signature at a later time point; and combining the provided signatures in the previous step to provide a progression marker indicative of the disease state in the subject. The invention furthermore relates to a pharmaceutical composition for use in treating a disease of the central nervous system (CNS). Also provided is a mobile device carrying out the methods of the invention.

The present invention relates to methods for providing the state of a disease in a subject comprising the steps of determining at least two parameters indicative of the state of a disease in the subject at a first time point; combining the determined parameters to provide a signature indicative of the disease state at the first time point; repeating the previous steps to provide at least one further signature at a later time point; and combining the provided signatures in the previous step to provide a progression marker indicative of the disease state in the subject. The invention furthermore relates to a pharmaceutical composition for use in treating a disease of the central nervous system (CNS). Also provided is a mobile device carrying out the methods of the invention.

Staging of diseases is a matter of ongoing research, in particular with respect to slowly progressing diseases such as for example diseases of the CNS. Staging is generally done based on biomarker profiles, which can be derived from varying sources. Most of the currently used biomarkers are determined in body samples, such as blood samples. For example, genetic markers for multiple sclerosis (MS) have been described by Mahurkar et al. (2017), Pharmacogenomics Journal 17(4):312-318. However, digital biomarkers have also been suggested as important source for determining disease stages. For example, a home-based pervasive computing system has been suggested for use in following changes of disease stage in patients with mild cognitive impairment or Alzheimer's disease; see Neil Thomas et al. Neurology Apr 2018, 90 (15 Supplement) P6.181.

Other studies have applied machine learning approaches for refining staging of diseases based on previously collected data. Such approaches have, for example, been described in US 2017/0091937 or WO 2019/200410.

The combination of digital biomarkers and genetic markers has been suggested for MS; see e.g. Cotsapas, C., & Mitrovic, M. (2018) Clinical & translational immunology, 7(6), e1018. doi:10.1002/cti2.1018; Uher et al. (2017) Mult Scler, 23(1):51-61; Hagens et al. (2016) Curr Opin Neurol.;29(3):229-36; Pardini et al. (2019) Current Opinion in Neurology; 32(3):358-364; and US 2019/0214140. However, the application has in a more recent review been found to be still unable to allow individual characterization and prediction; see Ziemssen et al. (2019) Journal of Neuroinflammation 16:272.

Bermel et al. (2013) Annals of Neurology vol 73, no. 1, pages 95-103 discloses a study to identify early predictors of long-term outcomes in patients. Further, US2019214140 discloses a method for assessing a cognition and movement disease or disorder in a subject.

Therefore, there is a need for reliable means and methods for providing the stage of a disease, in particular for diseases of the CNS such as MS.

The technical problem underlying the present invention is thus the provision of reliable means and methods providing the stage of a disease and their application in treatment decisions.

The above technical problem is solved by the embodiments provided herein and as characterized in the claims.

Thus, in a first aspect, the invention relates to a method for providing the state of a disease in a subject, the method comprising the steps of:
a) determining at least two parameters indicative of the state of a disease in the subject at a first time point;
b) combining the determined parameters in a) to provide a signature indicative of the disease state at the first time point;
c) repeating steps a) and b) to provide at least one further signature at a second time point;
d) combining the provided signatures in step c) to provide a progression marker indicative of the disease state in the subject.

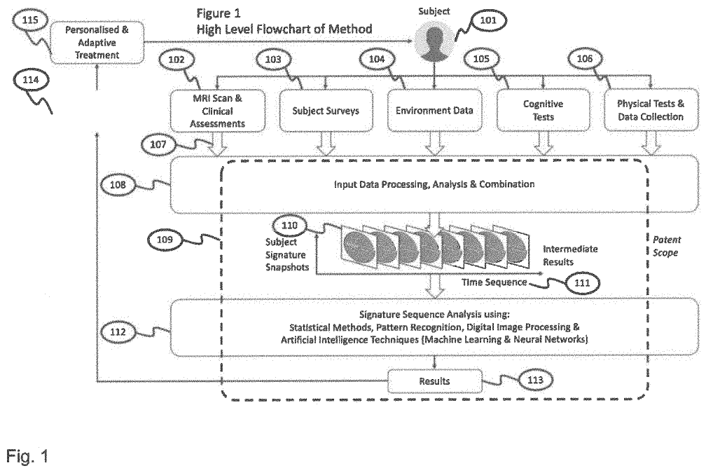

Accordingly, in a first step, the methods of the invention comprise a step of determining at least two parameters indicative of the state of a disease in the subject at a first time point. Within the present invention, the at least two parameters are the result of a data processing step using input data received from one or more sources; see (108) and (102) to (106) in FIG. 1. Upon sending and receiving, respectively, data from input sources, it becomes processed and analyzed, preferably including the removal of noise and/or analysis for any derived parameters and/or metrics it may produce. The method provided herein may comprise as a first step obtaining of at least two input parameters from the subject used for determining the at least two parameters indicative of the state of a disease in the subject. Preferably, the obtaining of the at least two input parameters is non-invasive.

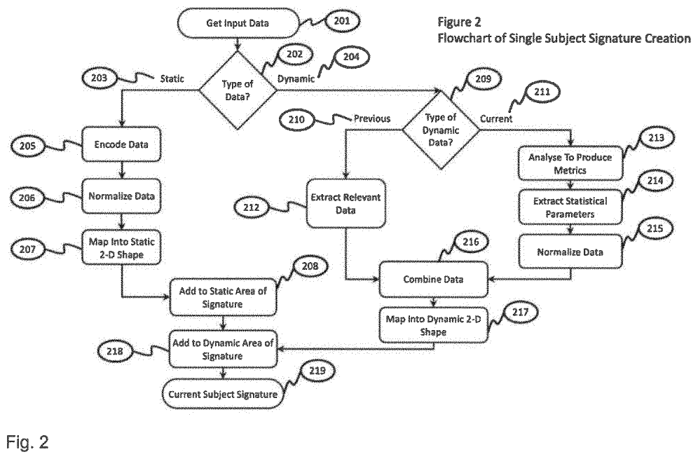

An exemplary processing method of input data is shown in FIGS. 2, (201) to (215). Accordingly, in some aspects of the invention, the input data (201) can be split (202) into static data (203) and dynamic data (204). Static data may be data that is measured infrequently such as for example data obtained from an imaging method, such as for example MRI or OCT. Dynamic data may be data captured more frequently, such as for example data captured by a mobile device, for example using an app on a mobile device. Dynamic data could result from activity testing or other tests provide herein. Static data may be encoded (205) into a format suitable for representation, such as for example representation in form of a digital image, for example a 10-bit grayscale number. The data may be normalized, for example in a manner to fit between 0.0 and 100.0 (206) representing approximately 1024 levels of a 10-bit grayscale image. The data may be mapped, for example into a static 2-D shape (207) such as a circle segment.

Dynamic data may be split (209) into data from the current period being represented by the signature, such as the last 2 weeks in the life of the subject, or into data from previous periods. The current signature may contain previous data and current data combined to form a moving window view of subject data representing for example the last 2, 3, 4, 5, 6, 7, 8, 9 or 10 periods of activities. In this respect, previous data may be extracted (212) from the previous signature for example. The current data may be analysed to produce key metrics from the raw measured data (214). These metrics may then be used to derive higher level statistical parameters (214) from the data set such as the distribution of data around a mean represented as the variance value. The data may be normalized (215) and/or combined with the previous data (216). The data may be mapped, for example onto a dynamic 2-D digital image shape such as a spiral (217).

Accordingly, the term “parameter” as used herein may be understood as “processed dynamic input data” or “processed static input data”, respectively.

In a second step of the method of the present invention, the determined parameters are combined to provide a signature indicative of the disease state at the first time point. Thus, processed dynamic and static data may be combined to form a signature of the disease state at the respective time point. For example, the static data may be added to a pre-designated static area of the final digital image (208) and the dynamic data may be added to a pre-designated dynamic area of the final digital image (218) to produce the subject signature (219).

In the methods of the present invention, at least two signatures are provided. That is, at least one further signature is provided at a second, preferably later, time point.

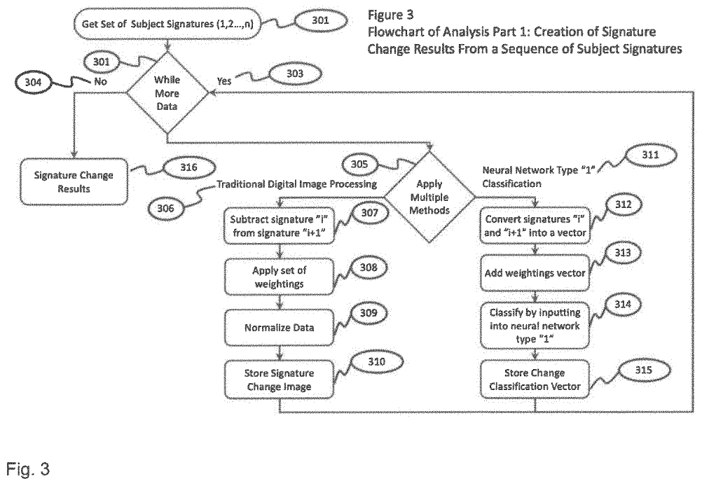

In a further step of the methods of the present invention, the provided signatures are combined to provide a progression marker indicative of the disease state in the subject; see FIG. 3. The methods thus involve processing of a time sequence of subject signatures (301). Preferably, two signatures are processed that are adjacent to each other in the time sequence of signatures (301). However, removing signatures before processing is also possible. As long as there are more signatures in the sequence (303) then two types of processing may be done (305) on each pair of signatures. For example, Traditional Digital Image Processing (306) may be used to perform actions such as subtracting one image from the other to find the differences (307). Other versions of this type of difference calculation may also be used. A set of weightings may be applied as some of the data may be more sensitive to changes than others (308). The difference data image may be normalized (309) before being stored as a Signature Change Image.

Additionally or alternatively, preferably additionally, a neural network may be used to classify the difference (311) having been previously trained up to interpret differences between subject signatures that are meaningful. This may be done by converting the pair of images into a single vector (312), adding a weighting vector for data areas that are more or less sensitive than others (313) and inputting into the neural network for a classification (314). The classification result is stored as a Change Classification Vector (315).

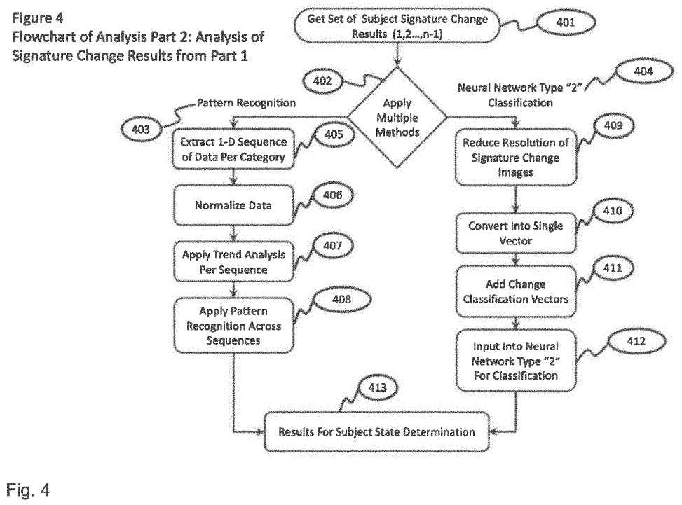

In a particular aspect of the invention, the methods of the invention further comprise a step of repeating steps a) to d) of the method of the invention in order to monitor disease progression in the subject based on the alteration of the progression marker provided in step d). In certain aspects, the method may be as shown in FIG. 4. That is, a signature change/alteration may be provided based on multiple, at least two, subject signatures. For example, there may be two separate processes applied (402) to the sequence and these processes may be performed in parallel or sequentially. Firstly, pattern recognition (403) using curve fitting, statistical analysis and/or other trend analysis techniques may be used to produce any clearly obvious trends or patterns from the data. This may be achieved by first extracting similar type data in 1-D sequences per category of test (405). This may be normalized so that all such 1-D sequences are in the same data range (406). The trend analysis techniques may be applied (407) and then pattern recognition (408) to identify for example multiple increasing trends and/or multiple decreasing trends across all of the sequences of data. In parallel or sequentially, the data may be fed into a neural network (404) that has been trained to recognize patterns of trends from a sequence of subject signature change/alteration results. Firstly, the change data images may be reduced in resolution in order to be able to send many images into a single neural network without the neural network being too large (409). Then multiple images may be combined into a single input vector for the neural network (410). Additional change classification vectors may be added (411) which were calculated by the first neural network used in part 1 of the analysis (FIG. 3). The data may be inputted into the neural network type 2 for classification (412). Finally, the trend analysis data and the neural network classification results may be used as input into a subject disease state determination (413), in particular determination of disease state progression. Accordingly, the present invention, in a further aspect, relates to a method for determining disease state progression comprising the steps provided herein as steps (a) to (e).

As shown in the further figures provided herein, the methods of the present invention provide various surprising advantages over methods of the prior art. For example, whereas prior art relies on the measurement of discrete, well described exercises, the methods of the invention allow the use of parameters measured passively (e.g. regular daily activities) and/or actively (e.g. by performing these exercises).

Within the present invention, it is preferred that the disease is a disease of the central nervous system (CNS), in particular a disease affecting motion, such as MS, Parkinson's disease, amyotrophic lateral sclerosis (ALS), epilepsy, Tourette, spinal muscular atrophy (SMA). Also included are diseases of the peripheral nervous systems and/or psychiatric diseases. Also included are neuromyelitis optica (NMO), stroke, Alzheimer's disease, depression, schizophrenia and the like.

In a particular aspect of the invention, the disease is multiple sclerosis (MS), in particular progressing MS, in particular relapsing-remitting MS with clinical disease activity, relapsing-remitting MS with disability progression, secondary progressive MS, secondary progressive MS with disability progression, primary progressive MS, or primary progressive MS with disability progression.

As detailed herein, the at least two parameters used in the methods of the invention may be provided based on data obtained from various sources before processing as described herein. In some aspects, parameters may be provided based on data obtained from one or more of:
(i) imaging techniques, in particular magnetic resonance imaging (MRI) and/or optical coherence tomography (OCT);
(ii) patient surveys regarding symptoms experienced by the subject;
(iii) environment data including weather information, vision tests, social interaction assessment, quality of life;
(iv) cognitive tests;
(v) physical tests, in particular testing motoric and/or fine motoric capabilities and/or function, walking, vision, sleep; and/or
(vi) biochemical marker determination, in particular as determined in a sample obtained from the subject, in particular blood, spinal cord fluid, cerebral spinal fluid, saliva and/or lymph.

The skilled person is aware of suitable imaging techniques available to provide representations of the interior of a body. Within the present invention, an imaging technique may be, but is not limited to, radiography, magnetic resonance imaging (MRI), in particular functional MRI, ultrasonography, elastography, photoacoustic imaging, tomography, echocardiography, functional near-infrared spectroscopy, magnetic particle imaging, diffuse optical topography, diffuse optical tomography, electrical impedance tomography, optoacoustic imaging, optical coherence tomography (OCT).

Patient surveys may also be used to provide data as basis for one ore more parameter(s) used in the methods of the invention. Patient surveys as used herein may aim to provide data related to symptoms experienced by the subject. Symptoms may, inter alia, be of physiological or mental nature.

Environmental data may also be collected as basis to provide one or more parameter(s) used in the methods of the invention. Environmental data may be of any kind, for example, weather information, temperature, humidity, season, location. It may also include vision tests, social interaction assessment and/or general quality of life.

Cognitive tests may also provide input data in the methods of the present invention. The skilled person is aware of various cognitive tests commonly employed in the assessment of disease states.

Physical tests may also be employed in the present invention. Such tests may, for example, relate to motoric and/or fine motoric capabilities and/or function, walking, vision, sleep. Tests may comprise walking, tight rope, climbing stairs, wobbler, U-turn, musical chairs, figures writing, screen to nose, cuddle a cloud, standing-up/sitting-down, level of activity, sleep, and/or heart rate. The skilled person is aware of standard tests employed in the disease state determination of various diseases, in particular MS. Common tests include 2-Minute Walking Test (2MWT), 5 U-Turn Test (5UTT), Static Balance test (SBT), eSDMT, CAG test, MSST test, Draw a Shape test, Squeeze a Shape test, Mood Scale Question test, MSIS-29, visual contrast and visual acuity tests (such as low contrast letter acuity or Ishihara test), and passive monitoring of all or a predetermined subset of activities of a subject performed during a certain time window. However, the invention as provided herein does not generally prefer the above tests and may alternatively or additionally include newly developed tests suitable for the desired purpose.

Biochemical marker may also be determined in the methods of the present invention. For example, biomarkers for MS have been described by Paul et al. (2019) Cold Spring Harbor Perspect Med 9(3). As a further biomarker gut microbiota may be used; see Pröbstel et al. (2018) Neurotherapeutics 15:126-134. Biomarkers may be determined in a sample obtained from the subject, in particular a sample obtained from blood, spinal cord fluid, cerebral spinal fluid, saliva and/or lymph. Biomarkers may also be obtained from non-invasive methods, for example electrophysiology.

Within the present invention, data may result from passive data collection or active data collection. That is, data may be obtained with our without direct input from the subject. Within the present invention, data, in particular passively collected data, may be continuously or quasi-continuously collected.

In some aspects of the invention, at least one parameter is determined by or using a mobile device. The mobile device may comprise a smartphone, smartwatch, wearable sensor, portable multimedia device or tablet computer. The mobile device, in particular the smartphone, smartwatch, wearable sensor, portable multimedia device or tablet computer, may actively or passively collect data from the user, for example using an app installed on the mobile device. It is preferred that the mobile device is able to transmit data to a system, for example a server or cloud based system, able to process and/or analyze the data collected by the mobile device.

In some aspects of the invention, the method steps may be repeated after a time interval determined based on the disease state. The methods of the invention allow determining the disease state based on two or more signatures. While more signatures are combined and the disease state is determined, it may be decided to alter the time interval between method step repetitions, signature provision, based on the disease state determined using the available signatures. For example, the time interval may be increased if only low progression of the disease state is determined. The interval may be decreased if fast progression of the disease state is determined. Notably, the interval does not necessarily have to be the same for determining one or more of the two or more parameters. That is, in some aspects of the invention, a newly provided parameter may be combined with an existing parameter to provide a signature. Additionally or alternatively, the data serving as basis for determining one or more of the two or more parameters may be obtained at different time points/intervals and be combined to provide one of the two or more parameters.

In some aspects of the invention, the methods comprises a step of selecting the parameters to be determined based on the disease state and/or disease progression. That is, in the methods of the invention, the parameters may be selected based on the disease state and/or the disease progression. For example, it may be decided to replace, add, remove or alter one or more of the parameters used to provide a signature based on the disease state and/or disease progression determined based on the previously provided signatures.

In some aspects of the invention, additionally or alternatively to replacing, adding, removing or altering one or more of the parameters, the parameters may be combined in a weighted manner. Similarly, it may be decided to combine signatures in a weighted manner in order to determine disease progression. For example, it may be decided to reduce weight of one or more previously obtained parameters/signatures with an increasing number of parameters/signatures. It may also be decided to adapt weight of one ore more parameters based on the type of disease, e.g. if a test is known or expected to provide higher/lower sensitivity.

Within the present invention, the methods may comprise the use of statistical methods, pattern recognition techniques, digital image processing, and/or artificial intelligence techniques, in particular machine learning and/or neural networks. In certain aspects of the invention, the used method/technique may be adapted based on the provided signatures/progression markers.

In a further aspect of the invention, a method for determining efficacy of therapy of a disease is provided, the method comprising the use of the method of the invention for providing the state of a disease in a subject, wherein therapy is determined to be efficient if the alteration of the progression marker is below a pre-determined threshold. That is, a subject may receive treatment of a disease with unknown or unclear efficacy of the treatment. The method for determining efficacy of therapy of a disease provided herein may be used to monitor efficacy of the treatment based on determining the disease state at a given time point and/or the progression of the disease state over a given time period. Based on the method provided herein, it may be decided that treatment is efficient. For example, it may be decided that treatment is efficient if the determined disease state is lower/less advanced than expected and/or progression of the disease is slower/reduced as compared to a previous prognosis. Alternatively, treatment may be determined as inefficient or having reduced efficiency if the disease state is higher/more advanced than expected and/or progression of the disease is faster/enhanced as compared to a previous prognosis. Accordingly, it is not required that treatment as received by the subject was previously shown to be effective. The method of the invention as provided herein may thus also be used for assessing treatment efficacy of newly developed or previously unknown treatment options. The method of the invention as provided herein may be used for screening of compounds for efficacy in the treatment of a disease.

In a further aspect, the invention relates to a pharmaceutical composition for use in treating a disease of the central nervous system (CNS), wherein treatment is initiated/adapted based on the disease state and/or progression of the disease determined by the methods of the invention.

In the present invention, treatment may, for example, comprise the use of interferon beta-1a, interferon beta-1b, an agent specifically binding to CD52, an agent specifically-binding to CD20, and/or an agent specifically binding to integrin.

Treatment as used herein can be understood to relate to the alleviation of symptoms associated with the disease.

Exemplary components of the pharmaceutical composition as provided herein are glatiramer, teriflunomide, fingolimod, dimethyl fumarate, siponimod, cladribine, alemtuzumab, mitoxantrone, ocrelizumab and/or natalizumab. However, the invention is not limited to the use of these compounds. Any compound potentially showing effectiveness in the treatment of a disease of the CNS, in particular MS, is encompassed by the present invention.

In a further embodiment, a method for treating a disease in a subject is provided, the method comprising determining the state of the disease in the subject and/or the progression of the disease in the subject according to the methods provided herein and administering to the subject an efficient amount of a therapeutic agent treating the disease or alleviating symptoms associated with the disease.

In a further aspect, the invention relates to a mobile device comprising a processor, at least one sensor, a database and software which is tangibly embedded in said device and, when running on said device, carries out the method of the invention. The mobile device may comprise or may be a smartphone, smartwatch, wearable sensor, portable multimedia device or tablet computer.

In a further aspect, the mobile device of the invention may be used for identifying a subject suffering from a disease of the CNS, in particular MS. The mobile device of the invention may also be used for monitoring progression of a disease of the CNS, in particular MS, and/or for monitoring treatment efficacy of a disease of the CNS, in particular MS.

In a further aspect, the invention relates to a system comprising a mobile device comprising at least one sensor and a remote device comprising a processor and a database as well as software which is tangibly embedded to said device and, when running on said device, carries out the method of the invention, wherein said mobile device and said remote device are operatively linked to each other.

The invention also provides a data carrier storing information with respect to the method of the invention, in particular a software able to carry out the method provided herein. The data carrier may be part of a server such as a cloud server.

The “subject” as used herein is preferably a mammal, more preferably a human subject. The subject may have previously been diagnosed with a disease. The subject may receive treatment of a disease or may have previously received treatment of a disease, in particular treatment of the diagnosed disease.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control.

While aspects of the invention are illustrated and described in detail in the drawings and foregoing description, such illustration and description are to be considered illustrative or exemplary and not restrictive. It will be understood that changes and modifications may be made by those of ordinary skill within the scope and spirit of the following claims. In particular, the present invention covers further embodiments with any combination of features from different embodiments described above and below.

The invention also covers all further features shown in the figures individually, although they may not have been described in the previous or following description. Also, single alternatives of the embodiments described in the figures and the description and single alternatives of features thereof can be disclaimed from the subject matter of the other aspect of the invention.

Furthermore, in the claims the word “comprising” does not exclude other elements or steps, and the indefinite article “a” or “an” does not exclude a plurality. A single unit may fulfill the functions of several features recited in the claims. The terms “essentially”, “about”, “approximately” and the like in connection with an attribute or a value particularly also define exactly the attribute or exactly the value, respectively. Any reference signs in the claims should not be construed as limiting the scope.

The present invention is also illustrated in some aspects by the following figures.

FIG. 1: High Level Flowchart of Method.

Subjects (101) have a variety of static and dynamic assessments and tests performed on them or by them divided into various categories. MRI Scans and Clinical Assessments (102) are performed typically annually in the hospital by the medical team. Subject Surveys (103) are performed using a smart phone app and include listing symptoms currently being experienced. Environment Data (104) includes weather information, vision tests, social interaction assessments and other external interactions that the Subject has. Cognitive Tests (105) test various brain functions and provide both raw test data and derived test data. Physical Tests & Data Collection (106) involves the Subject actively performing tests while a smart phone records data but also passive data collection while the Subject is moving about.

Data is collected and sent (107) and stored in a datacentre connected to the internet from where it can be easily accessed by a cloud software application implementing the method described (108-113). First the input data is processed to remove noise and analyze it for any derived parameters or metrics it may produce (108). The data is then processed and combined into a single Subject signature for each time period, for example once every 2 weeks (108). This results in a time sequence of Subject signature snapshots, each one representing a snapshot in time of the Subjects' disease progression for the 2-week period over which the tests were performed. These are the intermediate results (210). A combination of traditional statistical methods and artificial intelligence methods are performed in parallel (112) to produce the final results (113) as described in more detail in the following FIGS. (2-6).

FIG. 2: Flow Chart of Single Subject Signature Creation.

The input data (201) is first split (202) into static data (203) and dynamic data (204). Static data is data that is measured infrequently such as an MRI scan which might be taken only once per year. Dynamic data is data captured by an app on a mobile phone and could be taken frequently such as a walking test to measure gait every 2 weeks.

Static data is then encoded (205) into a format suitable for a digital image such as a 10-bit grayscale number. The data is then normalized to fit between 0.0 and 100.0 (206) representing approximately 1024 levels of a 10-bit grayscale image. The data is then mapped into a static 2-D shape (207) such as a circle segment.

Dynamic data is then also split (209) into data from the current period being represented by the signature, such as the last 2 weeks in the life of the Subject, or into data from previous periods. The current signature will contain previous data and current data combined to form a moving window view of Subject data representing for example the last 5 periods of activities.

Previous data can be extracted (212) from the previous signature for example. The current data is analysed to produce key metrics from the raw measured data (214). These metrics are then used to derive higher level statistical parameters (214) from the data set such as the distribution of data around a mean represented as the variance value. The data is then normalized (215) and combined with the previous data (216) before being mapped onto a dynamic 2-D digital image shape such as a spiral (217). The static data is added to a pre-designated static area of the final digital image (208) and the dynamic data is added to a pre-designated dynamic area of the final digital image (218) to produce the final current Subject signature (219).

FIG. 3: Flow Chart of Analysis Part 1

The figure shows part 1 of the data analysis which involves processing a time sequence of Subject signatures (301). In this sequence, this part of the analysis processes 2 signatures at a time that are adjacent to each other in the time sequence of signatures (301). As long as there are more signatures in the sequence (303) then 2 types of processing are done in parallel (305) on each pair of signatures. Traditional Digital Image Processing (306) is used to perform actions such as subtracting one image from the other to find the differences (307). Other versions of this type of difference calculation may also be used. A set of weightings is applied as some of the data may be more sensitive to changes than others (308). The difference data image is then normalized (309) before being stored as a Signature Change Image.

In parallel, a neural network is used to classify the difference (311) having been previously trained up to interpret differences between Subject signature images that are meaningful. This is done by converting the pair of images into a single vector (312), adding a weighting vector for data areas that are more or less sensitive than others (313) and inputting into the neural network for a classification (314). The classification result is stored as a Change Classification Vector (315).

FIG. 4: Flow Chart of Analysis Part 2

This figure shows the flow chart of the method component that deals with a time sequence of Subject signature change results (401). There are 2 separate processes applied (402) to the sequence and these processes are performed in parallel. Firstly, pattern recognition (403) using curve fitting, statistical analysis and other trend analysis techniques are used to produce any clearly obvious trends or patterns from the data. This is achieved by first extracting similar type data in 1-D sequences per category of test (405). This is normalized so that all such 1-D sequences are in the same data range (406). Then the trend analysis techniques are applied (407) and then pattern recognition (408) to identify for example multiple increasing trends or multiple decreasing trends across all of the sequences of data.

In parallel, the data is fed into a neural network (404) that has been trained to recognize patterns of trends from a sequence of Subject signature change results. Firstly, the change data images are reduced in resolution in order to be able to send many images into a single neural network without the neural network being too large (409). Then multiple images are combined into a single input vector for the neural network (410). Additional change classification vectors are added (411) which were calculated by the first neural network used in part 1 of the analysis (FIG. 3). The data is inputted into the neural network type 2 for classification (412). Finally, the trend analysis data and the neural network classification results are ready for input into a subject state determination (413).

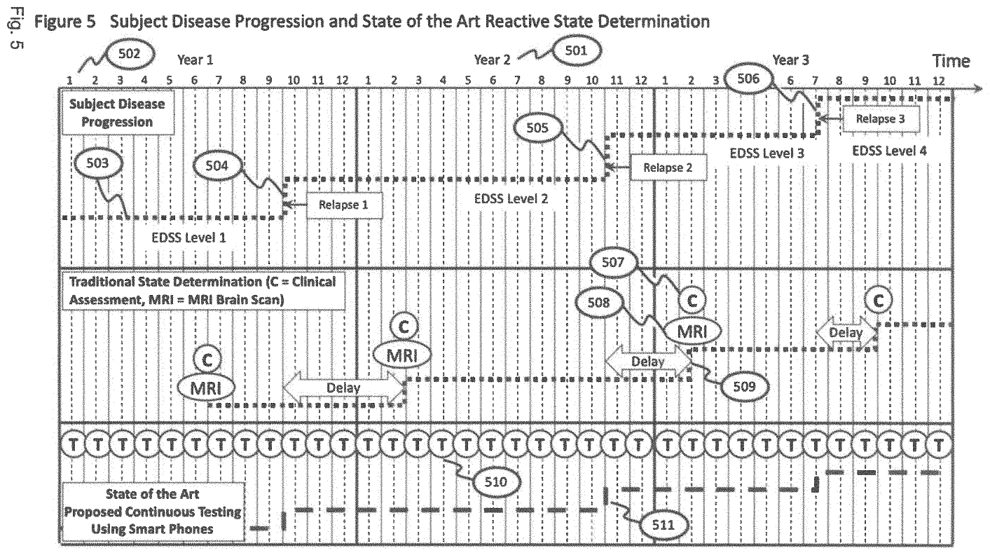

FIG. 5: Subject Disease Progression and State of the Art Reactive Diagnosis

This figure shows a timeline of 3 years (501) broken into months (502) and in the top section, displays the rise of a Subject's EDSS level as clinically assessed by their medical team (503) as the Subject progresses through several relapses (504,505,506) to move from level 1 to level 4.

The middle section illustrates how annual or bi-annual clinical assessments (507) and MRI scans (508) are often reactive in determining a change in EDSS level fora Subject and therefore any change in treatment or medication is reactive and has a built-in delay (509) after the Subject's actual disease progression.

The bottom section illustrates the use of some form of continuous testing using for example smart phone technology (510) in a monthly set of tests. The aim is to remove the delay and replicate more closely the actual Subject disease progression (511) curve.

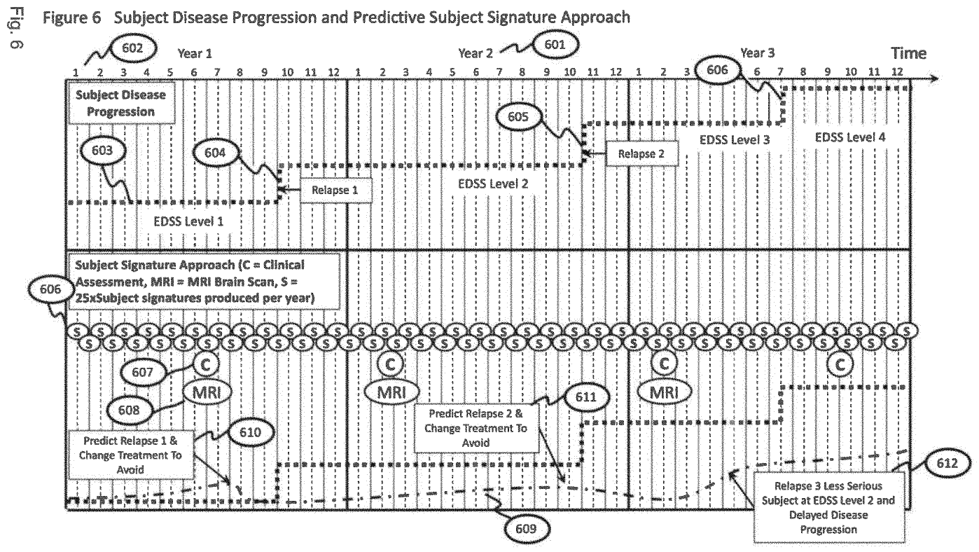

FIG. 6: Subject Disease Progression and Predictive Subject Signature Approach

This figure shows the same timeline (as in FIG. 5) of 3 years (601) broken into months (602) and in the top section, displays the rise of a Subject's EDSS level as clinically assessed by their medical team (603) as the Subject progresses through several relapses (604,605,606) to move from level 1 to level 4.

Using the method described in FIGS. 1 to 4, a Subject signature is generated every 2 weeks (606) alongside the same clinical assessment (607) timeline and MRI scan (608). However the higher resolution data and improved analysis achieved using the signature time sequences produce a more detailed view of Subject progression (609) that operates less like a step change and more like a continuous progression. The results from the signature time sequence analysis are able to predict the first relapse (610) and therefore adjust treatment to avoid it occurring. The same situation applies to Relapse 2 (611) and again the Subject avoids progressing their disease to EDSS level 2. Relapse 3 still occurs in this example (612) but it is less serious only moving the Subject to level 2 compared to level 4 in FIG. 5.

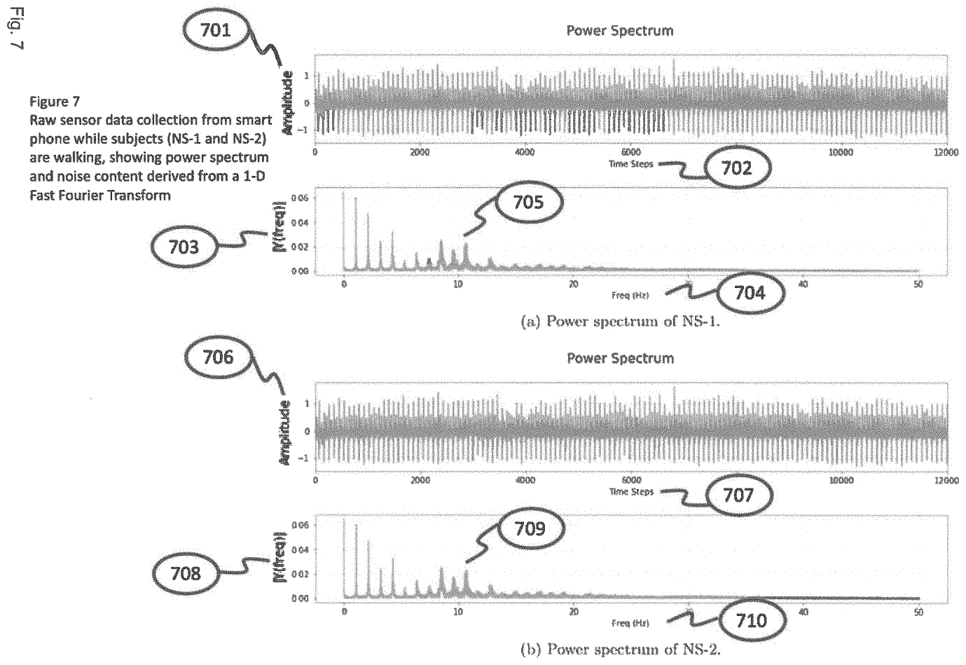

FIG. 7: Raw Sensor Data Collection From Smart Phone and Power Spectrum Analysis.

Data has been gathered from smart phone sensors carried by 2 different subjects while walking to analyze their gait. This data was taken from one of the accelerometers in the smart phone in the X direction. The raw data was plotted as an amplitude signal (701,706) against time steps (702,707) for each of the subjects. This raw data was then processed using a 1-Dimensional Fast Fourier Transform to produce a power spectrum showing the frequency content of the signals. This was plotted as the power density (703,708) against the frequency (704,710). The objective was to identify higher frequency noise in the signal (705,709) so that it can then be removed using a low pass filter on the data.

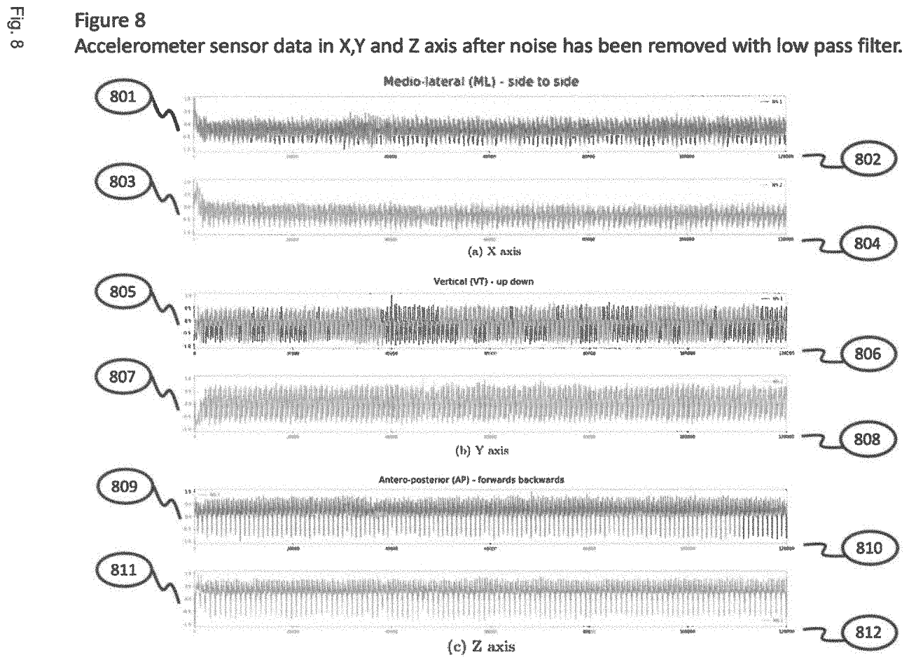

FIG. 8: Raw Sensor Data Collection From Smart Phone and Power Spectrum Analysis

This figure shows the accelerometer data taken from the smart phone for two subjects after it has been processed to remove higher frequency noise. The amplitude of the signal (801,803,805,807,809,811) was plotted against time steps (802,804,806,808,810,812). This cleaner set of signals was then ready to analyze to extract features and parameters for input into the subject signatures.

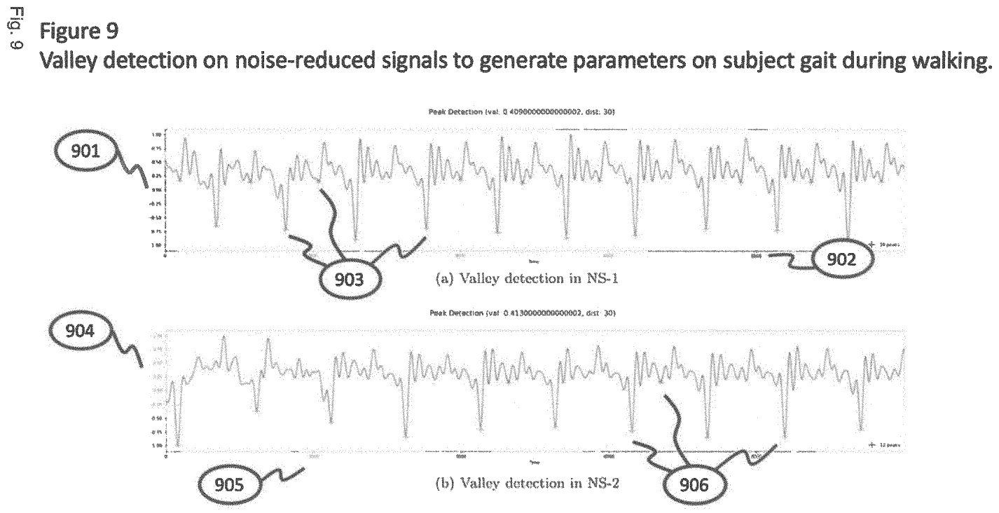

FIG. 9: Valley Detection on Noise-Reduced Signals

This figure illustrates how a valley detection algorithm was used to detect features of the noise-reduced accelerometer data gathered for the 2 subjects from their smart phones in one of the X direction. The amplitude of the signal (901,904) was plotted against time steps (902,905). Some examples of the valley points detected are shown in each plot (903,905).

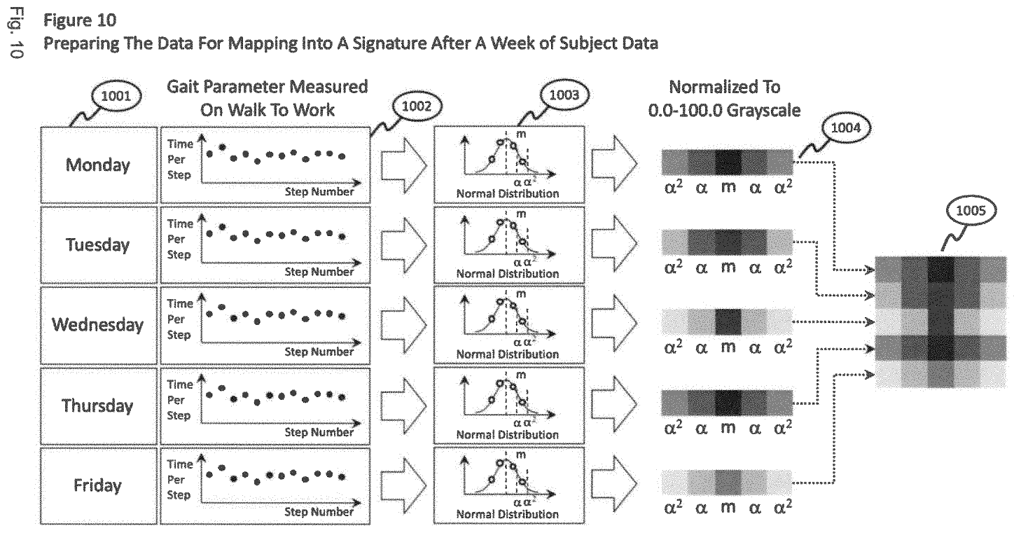

FIG. 10: Preparing the Data for Mapping to Signature

This figure illustrates how the data was mapped onto a set of pixels (1005) in a symmetrical 2-D pattern to enable the data to be mapped consistently into a larger signature pattern. For example, some data has been collected on the subject using their smart phone on each day of the week (1001) measuring their gait parameter of “time per step” (1002). Each day produces a set of data points that will have typically a normal distribution around a mean value. The data is fitted to a specific normal distribution and the mean, variance and variance-squared values are compute (1003). These values are normalized to a grayscale value between 0.0 and 100.0 representing 1000 levels or 10-bit resolution approximately in a grayscale digital image (1004). Note that the mean is placed in the centre while the variance values are replicated either side of the mean (1004). The 5 days of data are then gathered into a single group of values shown here as a 5×5 grid of 25 data points (1005).

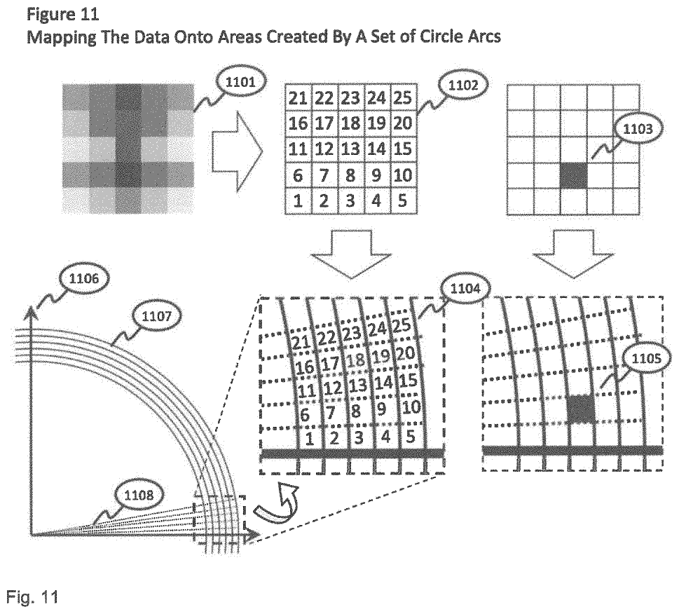

FIG. 11: Mapping The Data Onto Signature Areas

This figure shows how the matrix of data from FIG. 10 (1101) can then be mapped onto a curved area of the final signature (1106). In this example the data is mapped onto the areas formed by a set of concentric circles (1107) which have been divided into areas by lines emanating from the origin of the axes of the mapped space at 2 degree angles (1108) from the horizontal axis. The mapping of the 25 areas is then performed (1102,1104). So for each area in the matrix (1103), the value or colour is mapped into an equivalent space (1105).

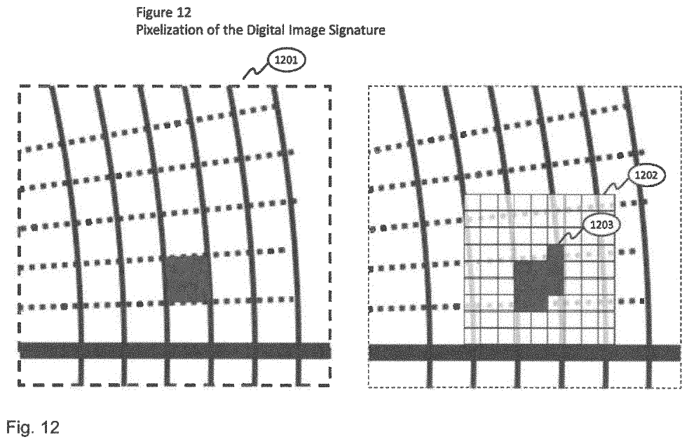

FIG. 12: Pixelization of the Digital Image Signature

The effect of pixelization is shown in this figure whereby a single value (1201) has been mapped into an area that is bounded by curved lines but which must be represented at a low resolution in order to keep the size of the signature to as small a number of values as possible. In this instance there the width of the area bounded by any two consecutive concentric circles is represented by 3 pixels (1202), so that the single data value (1201) is mapped onto 9 pixels in this instance (1203). The final pixel locations are recorded so that the mapping can be reversed.

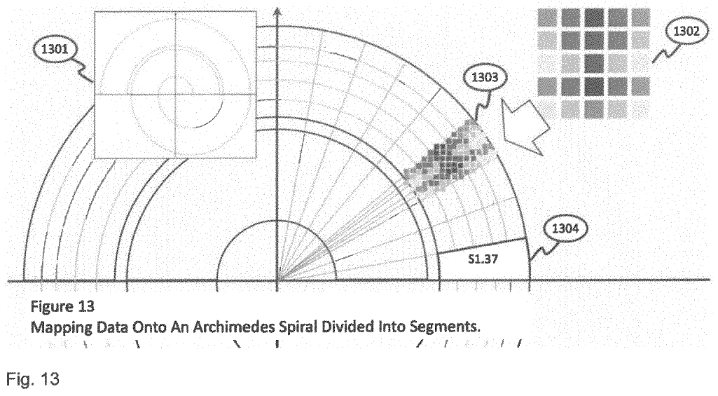

FIG. 13

The Figure shows an Archimedes Spiral (1301) which is used as a basis for mapping all of the subject parameters onto a single, expandable structure. The spiral is divided into segments of 10 degrees rotation (1304), making 36 per full rotation. Parameters from a specific type of test, for example, the walking test can be mapped into a segment (1302,1303) directly to an area of pixels. The outline of each individual area is used to decided whether a pixel is greater than 50% inside that area, and if it is >50% inside that area, all pixels within that area are given that parameter value. Therefore the 25 parameter values shown (1302) here are mapped into a larger number of actual pixels to create small areas of the same value (1303). The spiral shape can be repeated for different groupings of data types, for example inner spirals could be used for more static data, then move to environmental data, cognitive data and map physical data to the outermost spiral which will have the most pixels and therefore can contain the most parameters. Any variation on such a mapping could be contrived, for example using squares, rectangles, circles or ovals or any recognizable shape visible to the human eye. This allows for human interaction in both training of neural networks and presentation of specific signatures for review.

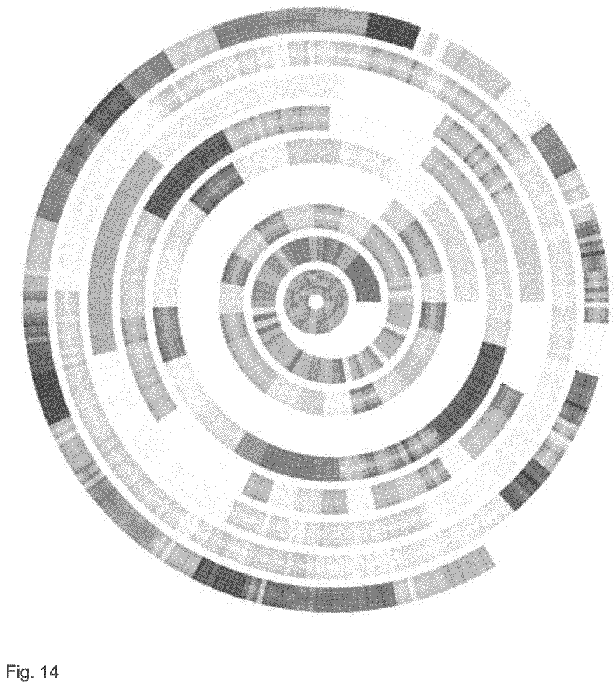

FIG. 14

Patient Digital Signature (PDS) constructed from real patient data. A summary of the data used to construct this PDS can be consulted at Appendix 1.

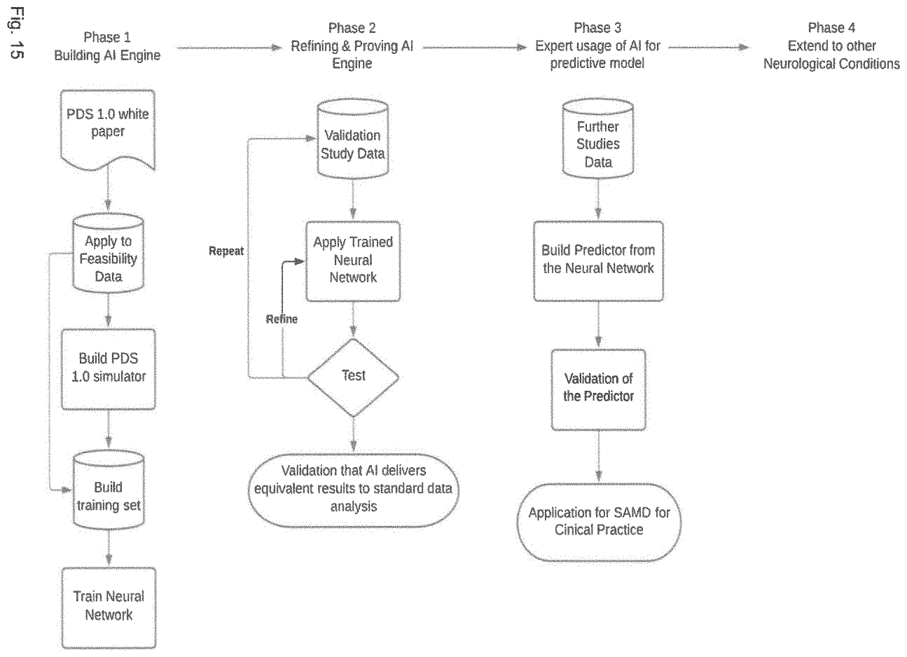

FIG. 15

R&D workflow in developing the PDS technology provided herein.

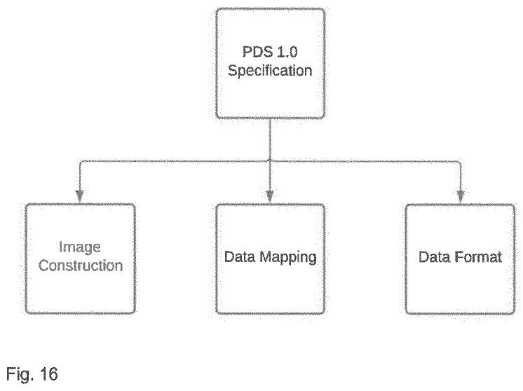

FIG. 16

Three components of the PDS specification.

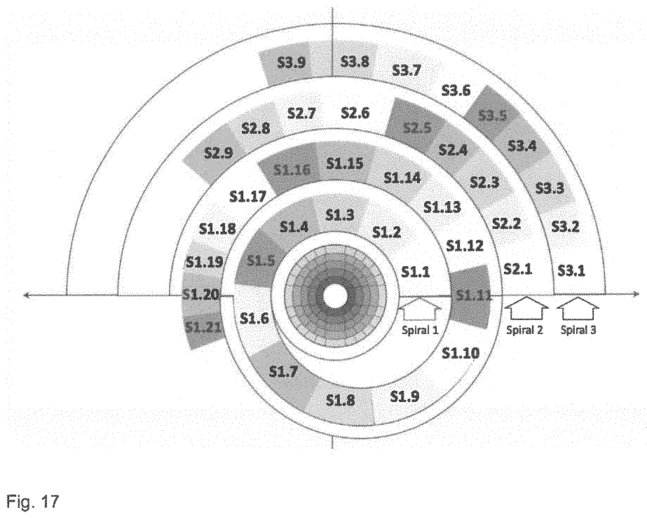

FIG. 17

Structure of the PDS.

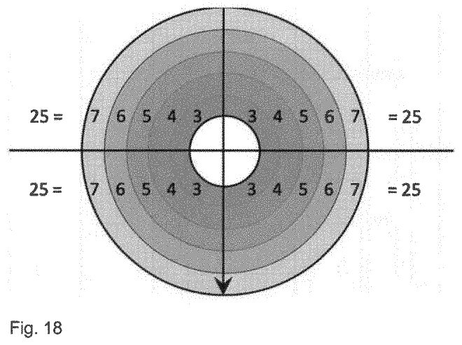

FIG. 18

Constructing an optimum data area inside the centre circle.

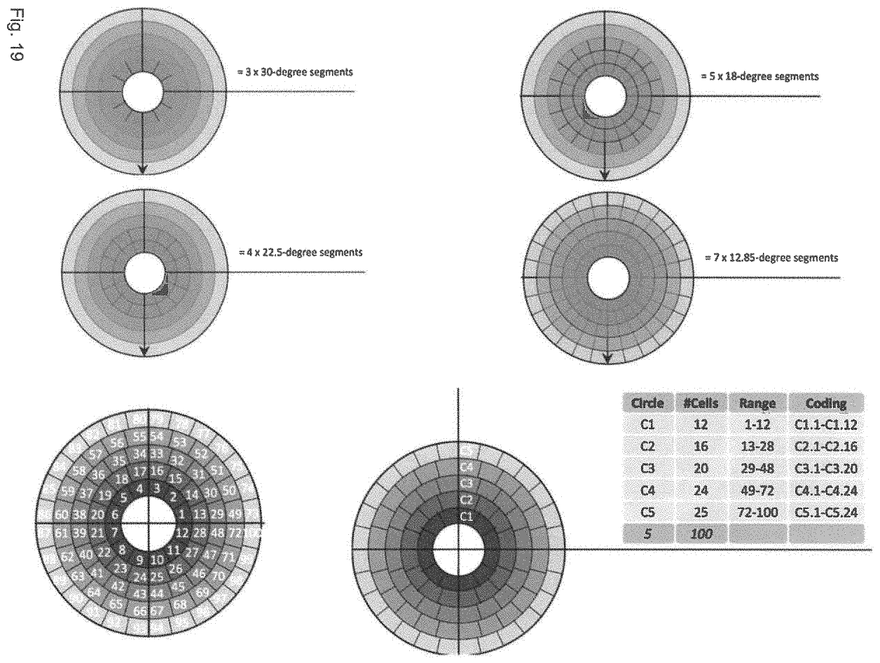

FIG. 19

Construction and coding of the centre circle data area.

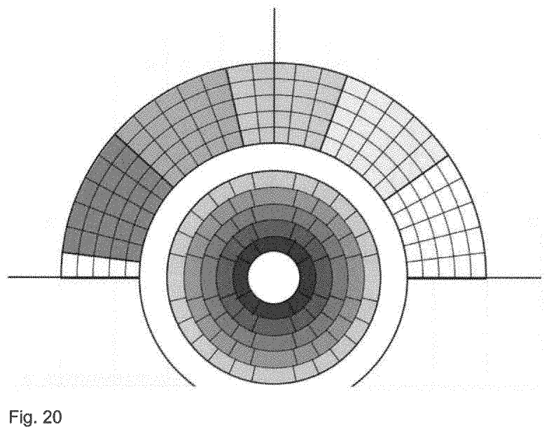

FIG. 20

Construction of the upper half of the first spiral using concentric semi-circles.

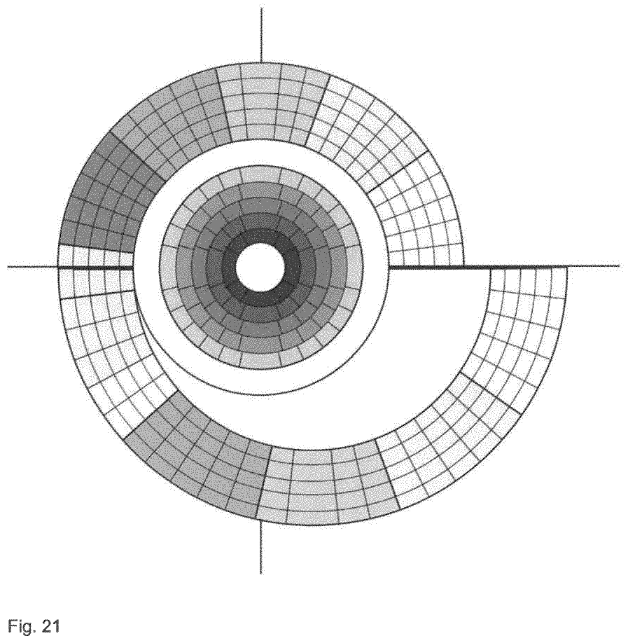

FIG. 21

Lower half of the spiral as it increases in radius.

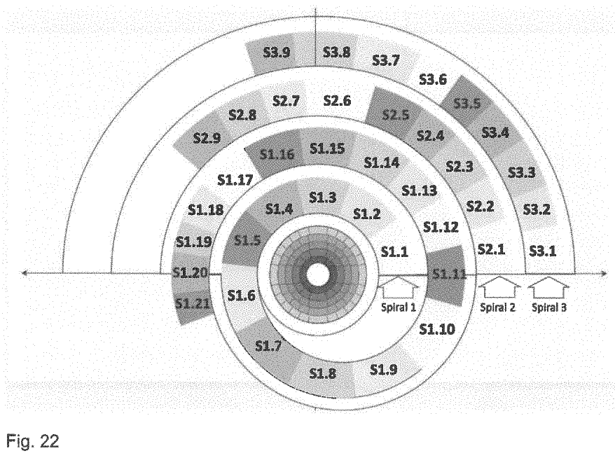

FIG. 22

Development of the spiral and division into 21 segments where each segment represents a specific patient challenge or block of data gathered over a period of time.

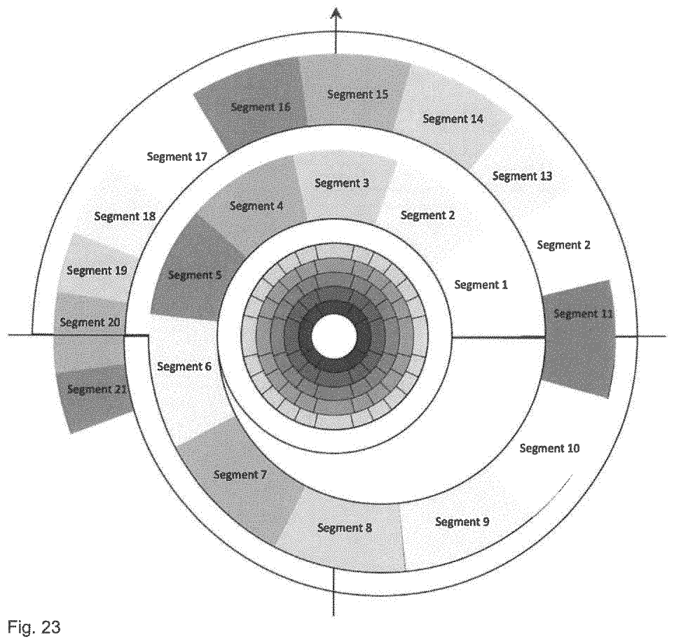

FIG. 23

The completed PDS showing 3 spirals and the numbering scheme for each spiral.

The invention is in the following further described by way of non-limiting examples.

### EXAMPLE 1

Provided herein is a first version of a new concept called a Patient Digital Signature (henceforth, PDS), and how it is constructed. A PDS is essentially a single grayscale image with an associated Data Map, that acts as an alternative representation of a set of data gathered from a patient over a set period of time. In particular, it is targeted for patients with chronic illnesses of the Central Nervous System (CNS), to solve the problem of how to handle large quantities of digital data that can be collected on the patient, including the use of apps on their smartphones over long periods of time. The PDS was initially applied to Multiple Sclerosis (MS) patients. As described herein the PDS can be used for various purposes including the diagnosis and/or monitoring of a disease, in particular disease of the CNS such as MS.

An example of a PDS constructed from real patient data obtained in an initial Feasibility Study with 45+ patients is shown in FIG. 14.

The data is organized into 4 elements:
1. A center circle—which will contain static data—e.g. data that does not change over a 6-month or annual period.
2. Spiral 1—which starts on the center horizontal line to the right of the center circle, and which contains survey and vision performance data gathered.
3. Spiral 2—which starts on the same center horizontal line but outside of Spiral 1, and which contains all motor-skill and cognitive data gathered.
4. Spiral 3—which starts on the same centre horizontal line but outside of Spiral 2, and which contains all physical data gathered.

FIG. 14 is a vector image generated using Python v.3 and therefore there are far more pixels used than are necessary to feed into a machine learning system. Therefore in the sections below it is shown how the minimum size PDS image is constructed in order to use more efficiently in a machine learning context, as provided herein.

### EXAMPLE 2

The goals of the PDS are summarised below which also outlines the problems that the PDS goals solve.

| No. | Goal | Problem Being Solved |
|---|---|---|
| 1 | Achieve a succinct | Potential overload of data for the medical team |
| | representation of a large set of | to look at or understand - potential for missing |
| | data gathered about a patient | important aspects or subtle trends in such a |
| | in a specific time period. | large dataset. |
| 2 | Complement standard data | Software As A Medical Device |
| | analysis techniques | |
| 3 | Minimise the size of the PDS | The size of the machine learning i/o and number |
| | image. | of processing nodes should be minimized where |
| | | possible to ensure performance and save on |
| | | processing and storage costs. |
| 4 | Ensure the PDS is reversible | Given a PDS file, it should be possible to build a |
| | so that it can be converted the | “reader” software programme that can map the |
| | image back into the original | values in the image back to their original values |
| | data by using a Data Map. | and know what that mapping is on a per file |
| | | basis. |
| 5 | Create several useful file | The PDS is both an image and a mapping, so in |
| | formats for PDS, such as text, | order to display the image, there should be |
| | jpeg lossless, png, or | some combination of a standard method to build |
| | proprietary binary so that long- | an image file plus additional mapping data for |
| | term storage size is optimised. | recovering the Data Map of the image. |

### EXAMPLE 3

This example presents a short summary of the PDS R&D workflow as this invention represents the first major deliverable in a series of phased R&D projects. FIG. 15 summarises the work being done in each of the 4 phases, which are then described in more detail below.

### 1. Phase 1

Provided herein is the first release of the PDS technique covering how the signature is constructed and which data is included. PDS is then being used to build the first real PDS files from the data produced in the current Feasibility Study. The next step is to build a software simulator that can generate quasi-realistic PDS files using a combination of the real PDS data from the Feasibility Study as a guide, and intelligent models of how that PDS might vary for different patients at different stages of their disease. The resulting large set of simulated PDS files will be used to train the first neural network to perform a variety of classifications that assist with patient tracking of disease progression.

### 2. Phase 2

In phase 2, the trained neural network will be applied to a larger real-world data set captured in a set of Validation Studies. In this phase, the trained neural network will be tested, refined and demonstrated with the aim of proving that it can replicate at a minimum the manual or automated standard analysis of the recorded data and how it relates to standard scales of patient condition measurement such as the EDSS scale and its derivatives.

### 3. Phase 3

The solid base produced in phase 2 should prove that the AI system can perform an accurate and equivalent measurement to any recognised technique for patient assessment. On this base, the Phase 3 work is to turn this measurement into a predictive measurement. This means that it is measuring the patient status so often and at high resolution that it can predict when a relapse might be about to happen. Another example of a prediction might be when the right moment is to change a patients' treatment to prevent a potential relapse even before any sign that a relapse is on the horizon for that patient. This predictive model would be proven in further real-world validation studies with intent of applying for certification as a medical device to be used in clinical practice.

### 4. Phase 4

The final phase is to then apply everything accomplished in MS to other neurological conditions such as Alzheimer's and Parkinson's for example.

### EXAMPLE 4

The PDS specification consists of 3 components as shown in FIG. 16. These components are:
Image Construction: These rules list the structural elements of the image part of the PDS to enable the automated construction of a PDS image down to the pixel level.
Data Mapping: This specifies what the measured patient data set is for this release of the PDS and how the measured patient data is mapped into the structural elements of the image described by the Image Construction specification rules.
Data Format: This component describes how the data should be modelled in a database for an active system using the PDS, and in what format the data could be retrieved over a remote interface such as RESTful Web API. It also describes how the data could be stored in a standalone file for long-term archiving purposes or sharing data in a minimised format between systems belonging to different groups or companies.

### EXAMPLE 5

In order to ensure a common language when specifying the Image Construction rules, a dictionary of definitions is provided herein below. These terms are used in the diagrams below to define how the image is constructed.

Dimensions=dimensions are provided in the unit of the size of a pixel, which could be the standard pixel size for a computer screen or represented as the minimum unit of digitalisation of the image in the form of a single square of one flat colour or value.

Pixel=the smallest unit of a PDS image, intended as the direct mapping onto a pixel of a computer screen.

Cell=a small group of pixels that are all adjacent to each other (either horizontally or vertically) and whose value or colour represents a single piece of measured patient data.

Cell Target Dimension=the minimum target dimensions of a cell, typically chosen to trade-off between overall data file size required and visibility issues to ensure that the digitisation process retains a reasonably clear shape in each spiral and that individual cells can be seen by human eyes on a particular screen, for example, PDS 1.0 has a Cell Target Dimension of (3×3) Pixels.

Segment=a segment of the arc area between two spiral lines drawn in the image, and which contains a 2-dimensional set of cells which is (m cells)×(s cells) in dimension, where the (m cells) represent data taken at one moment in time, and the (s cells) represent a time series.

Segment Number=an integer value, starting a ‘1’, that numbers the segments inside a spiral in the image, starting with the segment closest to the mid-horizontal line through the centre of the image (the zero x-axis of the cartesian coordinate space which has its origins at the centre point of the image), and increasing by +1 for each subsequent segment moving out towards the edges of the image.

Spiral=the entire area defined between two outer spiral lines drawn in the image.

Spiral Number=an integer value, starting at ‘1’, that numbers the spirals in an image starting with the spiral closest to the centre of the image and increasing by +1 for each subsequent spiral moving out towards the edges of the image.

Centre Circle=a circular area centered on the centre point of the image (the origin point of the cartesian coordinate space which has its origins at the centre point of the image). This is typically used to hold more static data that will stay the same over multiple signatures in a time sequence such as the patient's MRI scan dat, .a.

Centre Circle Radius=the radius given in Pixels for the Centre Circle.

Inter-Spiral Gap=the distance in Pixels between each Spiral as measured along the mid-horizontal line through the centre of the image (the zero x-axis of the cartesian coordinate space which has its origins at the centre point of the image).

Pixel Value (PV)=an integer between 0 and 1000 that represents a normalised data value between 0.00 and 100.00, whereby 0 is to be represented as the colour white, and 1000 is to be represented as the colour black. In binary format, this would be represented as a 10-bit value with 1024 levels. However the first 1000 levels are used, and the final 24 levels are not used in the PDS.

Normalized Data Value (NDV)=a floating point number between 0.00 and 100.00, where the range covered represents a normalized scale of ranges that maps exactly to the actual data range of some measured patient parameter.

Patient Data Value (PDV)=the actual value of patient measurement taken in the original units of that measurement, such as walking speed in km/hour.

Null Space Colour (NSC)=the value of the most extreme colour value used to fill the empty spaces and unused pixels in the image. This value is given in terms of an integer between 0 and 1000 that represents the grayscale colour of a 10-bit pixel colour. The NSC is ‘0’ or white for PDS 1.0. This means that blank spaces are filled in by some form of interpolation between any pixel that has a value and the NSC value.

Null Space Interpolation (NSI)=the type of interpolation used to fill in the colours of pixels in blank areas or unused pixels in the image. For example if the NSI is ‘Linear’, then each pixel between any specific pixel and the NSC pixel colour will be assigned a colour as closely as possible to a straight line interpolation between these 2 values (see example in diagrams below).

PDS Image Size (PIS)=the number of pixels in either the horizontal or vertical dimensions that the image is constructed of. The PDS image is a perfect square and so one value determines the size of the image.

Pixel Number (PN)=in certain situations it is more convenient to refer to a pixel by a set of coordinates. However if the cartesian coordinates are used with the natural origin at the centre then negative values will have to be entertained. Similarly if the digital image convention of using a reference system from the top left corner of the image is used, then it is counter to the cartesian system used to identify polygons, locations and dimensions in normal calculations. Therefore the PN consists of a pair of integers representing the number of pixels horizontally and vertically starting from the bottom left corner of the image as the (0,0) origin of the PN space.

Pixel Row (PR)=it is convenient to refer to a row of pixels, particularly when discussing how to turn a PDS image into a single vector for input into a neural network stage. Therefore a PR is an integer representing a row of pixels starting from the bottom row as ‘0’ and increasing by ‘+1’ for each row.

Pixel Column (PC)=it is convenient to refer to a column of pixels, particularly when discussing how to turn a PDS image into a single vector for input into a neural network stage. Therefore a PR is an integer representing a column of pixels starting from the bottom row as ‘0’ and increasing by ‘+1’ for each column.

### Image Construction

### 1. Top Level Structure

The structure of the PDS is shown in FIG. 17. There is a central circle, which contains 100 static data “cells”. Outside of this central circle there are 3 spirals, each divided into segments. The segments are numbered by which spiral they are in, followed by which sequential segment they are based on a starting point of the x-axis and rotating counter-clockwise as the segment number increases. This numbering can be summarised as SX.Y where X=the spiral number and Y=the segment number.

### 2. Centre Circle Structure

The centre circle contains static data and PDS allows for 100 data points to be stored. The design below is aimed at creating “cells” to store the data where each cell is a minimum of 3 pixels×3 pixels in shape size, and contains one value or one colour in that cell to represent the static data.

### 3. Spiral Construction

Spirals were constructed as concentric semi-circles with different sizes in the upper half of the image to the lower half of the image so that the spiral grows bigger on each turn. This is illustrated in the sequence of diagrams shown below starting with FIG. 20.

Note that the spiral is divided into segments (shown in different shades of grey), and within those segments there are 5 data cells across the width of each spiral.

Each segment can have up to 10 rows of data cells, or 5×10=50 data points.

### 4. Data Mapping

The following tables list the data embedded in PDS. The data of a patient are grouped in the following seven main/logical groups: imaging techniques, biochemical markers, surveys, vision tests, motor skill tests, cognitive tests, and physical tests. PDS contains one circle and three spirals that range from static data (like MRI), surveys, cognitive data and finally map physical data to the outermost spiral, which have the most pixels and therefore contain the most parameters. PDS comprises data currently used in clinical practice, .e.g. magnetic resonance imaging and common biochemical markers. Also, it includes the parameters extracted from the different tests performed by a patient when using DREAMS' application; e.g. parameters extracted from raw sensor signals.

PDS could also include environment data (e.g. data related to weather information, social interaction assessment, quality of life, etc), genetic markers, as well as data extracted from additional imaging techniques (e.g. optical coherence tomography) and biochemical markers. Furthermore, PDS could contain additional and refined parameters extracted from either the current DREAMS's tests or new tests that could be included into DREAMS' application.

Table 1 shows the parameters extracted from MRI images, which evaluates the inflammatory and neurodegenerative processes in the brain and spinal cord; it is the most commonly used technique for the evaluation of patients with MS.

TABLE 1
Parameters extracted from imaging techniques.

| | | |
|---|---|---|
| Segment | | |
| number | Imaging techniques | Parameter |

| | |
|---|---|
| Magnetic Resonance | Number of T2-lesions |
| Imaging | Volume of T2-lesions |
| | Number of Gadolinium |
| | enhancing lesions |
| | Volume of Gadolinium |
| | enhancing lesions |

Table 2 portrays the data related to biochemical markers that are included in PDS. For example, oligoclonal bands are found in nearly all patients with clinically definite MS (they occur in the analysis of cerebrospinal fluid), so it is a strong indicator of intrathecal antibody synthesis.

TABLE 2
Parameters extracted from biochemical markers.

| Segment | | |
|---|---|---|
| number | Biochemical markers | Parameter |
| | Blood and cerebrospinal | Oligoclonal IgG bands in |
| | Fluid | cerebrospinal fluid |
| | | JC virus-antibody status |
| | Neurophysiology | Visual evoked potentials |
| | | Somato-sensory evoked potentials |
| | | Motor evoked potentials |

Table 3 describes the data related to surveys taken by a patient (surveys included into DREAMS's application), which measure, for example, the severity of fatigue and its effect on a person's activities and lifestyle in patients with a variety of disorders.

TABLE 3
Parameters extracted from surveys.

| Segment | | |
|---|---|---|
| number | Surveys | Parameter |
| S1.1 | Fatigue Severity Scale | A parameter represents a response to |
| | | one question of the survey. This survey |
| | | has 9 parameters. |
| S1.2 | Multiple Sclerosis Walking Survey (MSWS-12) | A parameter represents a response to |
| | | one question of the survey. This survey |
| | | has 12 parameters. |
| S1.3 | Symptom trackers | A parameter represents a response to |
| | | one question of the survey. This survey |
| | | has 16 parameters. |
| S1.4 | Multiple Sclerosis Impact Scale (MSIS-29) | A parameter represents a response to |
| | | one question of the survey. This survey |
| | | has 29 parameters. |

Next tables describe the parameters calculated from the DREAMS' vision, motor skill, cognitive and physical tests. In the tables, examples of the raw and normalized values of the parameters for a real patient are given. The minimum and maximum values of the parameters have been calculated by means of observing two populations of subjects, namely a population of healthy subjects (from early lab-testing conducted in Córdoba, Spain) and a population of MS patients (from the feasibility study). Minimum and maximum values were calculated by subtracting and adding, respectively, at most four times the standard deviation from the mean of the joint population; bear in mind that the upper and lower limit values for each parameter should be refined when a larger population of MS patients will be available.

Table 4 shows the parameters extracted from DREAMS' vision tests, which assess fluctuation in quality of (contrast) vision over time, impacting daily living. Furthermore, from these tests are extracted several sensor-based parameters that measure the tremor level.

TABLE 4
Parameters extracted from vision tests.

| Segment | | Parameter | Example value of a | Normalised value |
|---|---|---|---|---|
| number | Vision tests | (measure unit) | real patient | (0-100) |
| S1.5 | Vision Acuity | Score left (unit-less) | 20 | 96.77 |
| S1.6 | | Score right (unit- | 20 | 96.77 |
| | | less) | | |
| S1.7 | | Normalized Path | 0.08 | 24.13 |
| | | Length - Medio | | |
| | | Lateral (unit-less) | | |
| S1.8 | | Normalized Path | 0.09 | 20.5 |
| | | Length -Vertical | | |
| | | (unit-less) | | |
| S1.9 | | Normalized Path | 0.01 | 20 |
| | | Length - Antero | | |
| | | Posterior (unit-less) | | |
| S1.10 | | Mean velocity-Medio | 0.03 | 10 |
| | | lateral (m/s) | | |
| S1.11 | | Mean velocity- | 0.01 | 2.5 |
| | | vertical (m/s) | | |
| S1.12 | | Mean velocity-Antero | 0.13 | 13.89 |
| | | Posterior (m/s) | | |
| S1.13 | | Peak power | 0.002 | 20 |
| | | acceleration (m²/s⁴) | | |
| S1.14 | | Tremor acceleration | 0.02 | 33.33 |
| S1.15 | | Total power | 0.29 | 16.11 |
| | | acceleration | | |
| S1.16 | | Peak power | 0.003 | 10 |
| | | gyroscope | | |
| S1.17 | | Tremor gyroscope | 0.02 | 8 |
| S1.18 | | Total power | 0.31 | 10.03 |
| | | gyroscope | | |
| S1.19 | Vision Contrast | Score left (unit-less) | 2 | 86.95 |
| S1.20 | | Score right (unit- | 2 | 86.95 |
| | | less) | | |
| S1.21 | | Normalized Path | 0.11 | 29.6 |
| | | Length - Medio | | |
| | | Lateral (unit-less) | | |
| S1.22 | | Normalized Path | 0.04 | 2.38 |
| | | Length -Vertical | | |
| | | (unit-less) | | |
| S1.23 | | Normalized Path | 0.02 | 14.28 |
| | | Length - Antero | | |
| | | Posterior (unit-less) | | |
| S1.24 | | Mean velocity-Medio | 0.03 | 9.37 |
| | | lateral (m/s) | | |
| S1.25 | | Mean velocity- | 0.01 | 2 |
| | | Vertica (m/s)l | | |
| S1.26 | | Mean velocity-Antero | 0.18 | 18.6 |
| | | Posterior (m/s) | | |
| S1.27 | | Peak power | 0.002 | 6.66 |
| | | acceleration (m²/s⁴) | | |
| S1.28 | | Tremor acceleration | 0.01 | 14.28 |
| S1.29 | | Total power | 0.04 | 2.4 |
| | | acceleration | | |
| S1.30 | | Peak power | 0.003 | 7.5 |
| | | gyroscope | | |
| S1.31 | | Tremor gyroscope | 0.02 | 10 |
| S1.32 | | Total power | 0.29 | 9.36 |
| | | gyroscope | | |

Table 5 describes the parameters extracted from DREAMS' motor skill tests, which assess tremor/cerebellar dysfunction, fine distal motor manipulation, motor control and impaired hand-eye coordination.

TABLE 5
Parameters extracted from motor-skill tests.

| Segment | | | Example value of a | Normalised value |
|---|---|---|---|---|
| number | Motor Skill Tests | Parameter | real patient | (0-100) |
| S2.1 | Catch A Cloud | Score (unit-less) | 44 | 73.33 |
| S2.2 | | Normalized Path | 0.18 | 39.28 |
| | | Length - Medio | | |
| | | Lateral (unit-less) | | |
| S2.3 | | Normalized Path | 0.18 | 41.9 |
| | | Length -Vertical | | |
| | | (unit-less) | | |
| S2.4 | | Normalized Path | 0.05 | 16.67 |
| | | Length - Antero | | |
| | | Posterior (unit-less) | | |
| S2.5 | | Mean velocity-Medio | 0.1 | 20 |
| | | lateral (m/s) | | |
| S2.6 | | Mean velocity- | 0.13 | 21.66 |
| | | vertical (m/s) | | |
| S2.7 | | Mean velocity-Antero | 0.45 | 22.22 |
| | | Posterior (m/s) | | |
| S2.8 | | Peak power | 0.01 | 33.33 |
| | | acceleration (m²/s⁴) | | |
| S2.9 | | Tremor acceleration | 0.05 | 25 |
| | | (m/s²) | | |
| S2.10 | | Total power | 0.37 | 37 |
| | | acceleration (m²/s⁴) | | |
| S2.11 | | Peak power | 0.03 | 15 |
| | | gyroscope (rad²/s²) | | |
| S2.12 | | Tremor gyroscope | 0.15 | 16.12 |
| | | (rad/s) | | |
| S2.13 | | Total power | 1.30 | 18.45 |
| | | gyroscope (rad²/s²) | | |
| S2.14 | Screen to Nose | Number of touches | 10 | 58.33 |
| | | (unit-less) | | |
| S2.15 | | Touch time (s) | 0.88 | 21.48 |
| S2.16 | | Touch velocity (m/s) | 0.75 | 11.4 |
| S2.17 | | Stretch time (s) | 0.73 | 15.9 |
| S2.18 | | Stretch velocity (m/s) | 0.68 | 9.6 |
| S2.19 | | Touch jerk swayness | 0.6 | 10.9 |
| | | (m²/s⁵) | | |
| S2.20 | | Stretch jerk | 0.26 | 4.7 |
| | | swayness (m²/s⁵) | | |
| S2.21 | | Touch tremor | 0.29 | 35.44 |
| | | acceleration (m/s²) | | |
| S2.22 | | Touch tremor | 1.12 | 41.5 |
| | | gyroscope (rad/s) | | |
| S2.23 | | Stretch tremor | 0.21 | 25 |
| | | acceleration (m/s²) | | |
| S2.24 | | Stretch tremor | 0.88 | 32.78 |
| | | gyroscope (rad/s) | | |

Table 6 describes the parameters extracted from DREAMS' cognitive tests, which assess fluctuation processing of information over time, impacting daily living. Furthermore, from these tests are extracted several sensor-based parameters that measure the tremor level.

TABLE 6
Parameters extracted from cognitive tests.

| Segment | | | Example value of a | Normalised value |
|---|---|---|---|---|
| number | Cognitive tests | Parameter | real patient | (0-100) |
| S2.25 | Symbol Digit Modalities | Normalized Path | 0.09 | 22.22 |
| | Test (m-SDMT) | Length - Medio | | |
| | | Lateral (unit-less) | | |
| S2.26 | | Normalized Path | 0.13 | 26 |
| | | Length -Vertical | | |
| | | (unit-less) | | |
| S2.27 | | Normalized Path | 0.02 | 12.5 |
| | | Length - Antero | | |
| | | Posterior (unit-less) | | |
| S2.28 | | Mean velocity-Medio | 0.09 | 15 |
| | | lateral (m/s) | | |
| S2.29 | | Mean velocity- | 0.03 | 6 |
| | | vertical (m/s) | | |
| S2.30 | | Mean velocity-Antero | 0.16 | 9.5 |
| | | Posterior (m/s) | | |
| S2.31 | | Peak power | 0.003 | 30 |
| | | acceleration (m²/s⁴) | | |
| S2.32 | | Tremor acceleration | 0.02 | 40 |
| | | (m/s²) | | |
| S2.33 | | Total power | 0.36 | 45 |
| | | acceleration (m²/s⁴) | | |
| S2.34 | | Peak power | 0.002 | 10 |
| | | gyroscope (rad²/s²) | | |
| S2.35 | | Tremor gyroscope | 0.02 | 10 |
| | | (rad/s) | | |
| S2.36 | | Total power | 0.29 | 7.01 |
| | | gyroscope (rad²/s²) | | |

Table 7 shows the parameters extracted from DREAMS' physical tests. For example, the Two min-Walk and U-turns tests provide an indication of the PwMS' walking and gait difficulties, commonly caused by weakness, spasticity, loss of balance, sensory deficit and fatigue. Climbing stairs test measures functional strength, balance and agility through ascending and descending steps. Tight Rope addresses the level of balance problems. Finally, Wobbler assesses if the person can keep arms extended for at least 10 seconds without descending; turning of the hand/pulse is identified as a sensitive early indication of MS.

TABLE 7
Parameters extracted from physical tests.

| Segment | | | Example value of a | Normalised value |
|---|---|---|---|---|
| number | Physical tests | Parameter | real patient | (0-100) |
| S3.1 | Tight Rope | Normalized Path | 0.28 | 23.64 |
| | | Length-Medio lateral | | |
| | | (unit-less) | | |
| S3.2 | | Normalized Path | 0.13 | 20 |
| | | Length-Anterior | | |
| | | Posterior (unit-less) | | |
| S3.3 | | Jerk swayness | 0.0013 | 0.13 |
| | | (m²/s⁵) | | |
| S3.4 | Wobbler | Normalized Path | 0.25 | 15.09 |
| | | Length - Medio | | |
| | | Lateral (unit-less) | | |
| S3.5 | | Normalized Path | 0.32 | 17.07 |
| | | Length -Vertical | | |
| | | (unit-less) | | |
| S3.6 | | Normalized Path | 0.13 | 40 |
| | | Length - Antero | | |
| | | Posterior (unit-less) | | |
| S3.7 | | Mean velocity-Medio | 0.03 | 2.5 |
| | | lateral (m/s) | | |
| S3.8 | | Mean velocity- | 0.01 | 0.62 |
| | | Vertica (m/s) | | |
| S3.9 | | Mean velocity-Antero | 0.17 | 5.31 |
| | | Posterior (m/s) | | |
| S3.10 | | Peak power | 0.01 | 1.25 |
| | | acceleration (m²/s⁴) | | |
| S3.11 | | Tremor acceleration | 0.03 | 16.67 |
| | | (m/s²) | | |
| S3.12 | | Total power | 0.07 | 7 |
| | | acceleration (m²/s⁴) | | |
| S3.13 | | Peak power | 0.01 | 5 |
| | | gyroscope (rad²/s²) | | |
| S3.14 | | Tremor gyroscope | 0.03 | 6 |
| | | (rad/s) | | |
| S3.15 | | Total power | 0.15 | 4.3 |
| | | gyroscope (rad²/s²) | | |
| S3.16 | Musical Chairs | Number of squats | 10 | 25.9 |
| | | (unit-less) | | |
| S3.17 | | Time to sit (s) | 1.03 | 15.14 |
| S3.18 | | Velocity to sit (m/s) | 0.46 | 8.8 |
| S3.19 | | Jerk swayness sit | 0.08 | 3.63 |
| | | (m²/s⁵) | | |
| S3.20 | | Time to stand (s) | 1.4 | 8.99 |
| S3.21 | | Velocity to stand | 0.34 | 12.90 |
| | | (m/s) | | |
| S3.22 | | Jerk swayness stand | 0.12 | 5 |
| | | (m²/s⁵) | | |
| S3.23 | U-turns | Number of u-turns | 7 | 40 |
| | | (unit-less) | | |
| S3.24 | | Time to u-turns (s) | 1.08 | 6.3 |
| S3.25 | | Velocity of u-turns | 166.52 | 74.42 |
| | | (degrees/s) | | |
| S3.26 | Climbing Stairs | Number of steps | 27 | 55 |
| | | (unit-less) | | |
| S3.27 | | Cadence (steps/s) | 2.15 | 71.66 |
| S3.28 | | Step time (s) | 0.46 | 30 |
| S3.29 | | Step regularity | 0.76 | 70 |
| | | (correlation - unit | | |
| | | less) | | |
| S3.30 | | Step dynamic time | 0.74 | 48 |
| | | warping (similarity - | | |
| | | unit less) | | |
| S3.31 | | Step jerk swayness | 0.08 | 53.33 |
| | | (m²/s⁵) | | |
| S3.32 | | Number of strides | 13 | 55 |
| | | (unit-less) | | |
| S3.33 | | Stride time (s) | 0.93 | 30.71 |
| S3.34 | | Stride regularity | 0.72 | 65 |
| | | (correlation - unit | | |
| | | less) | | |
| S3.35 | | Stride dynamic time | 0.74 | 48 |
| | | warping (similarity - | | |
| | | unit less) | | |
| S3.36 | | Stride jerk swayness | 0.16 | 53.33 |
| | | (m²/s⁵) | | |
| S3.37 | | Gait Symmetric | 0.95 | 95 |
| | | (percentage - unit | | |
| | | less) | | |
| S3.38 | Two-Min Walk | Number of steps | 239 | 85.31 |
| | | (unit-less) | | |
| S3.39 | | Cadence (steps/s) | 2.05 | 68.33 |
| S3.40 | | Step time (s) | 0.49 | 7.03 |
| | | Step regularity | 0.95 | 94.73 |
| | | (correlation - unit | | |
| | | less) | | |
| S3.41 | | Step dynamic time | 0.88 | 76 |
| | | warping (similarity - | | |
| | | unit less) | | |
| S3.42 | | Step jerk swayness | 0.04 | 6.4 |
| | | (m²/s⁵) | | |
| S3.43 | | Number of strides | 119 | 84.89 |
| | | (unit-less) | | |
| S3.44 | | Stride time (s) | 0.97 | 6.85 |
| S3.45 | | Stride regularity | 0.93 | 92.2 |
| | | (correlation - unit | | |
| | | less) | | |
| S3.46 | | Stride dynamic time | 0.82 | 64 |
| | | warping (similarity - | | |
| | | unit less) | | |
| S3.47 | | Stride jerk swayness | 0.09 | 5.03 |
| | | (m²/s⁵) | | |
| S3.48 | | Gait Symmetric | 0.96 | 96 |
| | | (percentage - unit | | |
| | | less) | | |

### EXAMPLE 6

The following tables show a summary of the data from a real MS patient. These data were used to generate the PDS given as an example herein above.

TABLE 8
Parameters extracted from the surveys.

| Surveys | Response given to each question |
|---|---|
| Fatigue Severity Scale | 7, 7, 7, 7, 7, 7, 7, 4, 4 |
| Multiple Sclerosis Walking | 3, 3, 4, 4, 3, 3, 4, 5, 1, 3, 4, 2 |
| Survey (MSWS-12) | |
| Symptom trackers | 3, 4, 1, 3, 3, 3, 4, 1, 3, 3, 3, 3, |
| | 2, 1, 3, 2 |

TABLE 9
Parameters extracted from the Vision Acuity test. NPL-ML: mean normalized path
length - mean medio lateral, NPL-V: mean normalized path length-Vertical, NPL-AP:
mean normalized path length-Antero Posterior, Vel-ML: mean velocity-Medio lateral,
Vel-V: mean velocity-Vertical, Vel-AP: mean velocity-Antero Posterior, PPA:
mean peak power acceleration, TA: mean tremor acceleration, TPA: mean
total power acceleration, PPG: mean peak power gyroscope, TG: mean
tremor gyroscope, TPG: mean total power gyroscope.

| | Score | Score | NPL- | NPL- | NPL- | Vel- | Vel- | Vel- | | | | | | |
|---|---|---|---|---|---|---|---|---|---|---|---|---|---|---|
| Rep. | left | right | ML | V | AP | ML | V | AP | PPA | TA | TPA | PPG | TG | TPG |
| 1 | 32 | 32 | 0.102 | 0.065 | 0.021 | 0.032 | 0.070 | 0.163 | 0.002 | 0.013 | 0.299 | 0.006 | 0.054 | 1.291 |
| 2 | 32 | 40 | 0.041 | 0.034 | 0.019 | 0.047 | 0.072 | 0.158 | 0.004 | 0.027 | 0.561 | 0.004 | 0.043 | 0.746 |
| 3 | 32 | 40 | 0.052 | 0.036 | 0.010 | 0.035 | 0.032 | 0.081 | 0.002 | 0.026 | 0.506 | 0.004 | 0.055 | 1.084 |
| 4 | 40 | 40 | 0.124 | 0.105 | 0.018 | 0.040 | 0.040 | 0.145 | 0.003 | 0.027 | 0.521 | 0.014 | 0.093 | 1.920 |
| 5 | 32 | 40 | 0.096 | 0.101 | 0.013 | 0.034 | 0.025 | 0.109 | 0.002 | 0.012 | 0.224 | 0.003 | 0.025 | 0.436 |

Table A2. Parameters extracted from the Vision Acuity test. NPL-ML: mean normalized path length-mean medio lateral, NPL-V: mean normalized path length-Vertical, NPL-AP: mean normalized path length-Antero Posterior, Vel-ML: mean velocity-Medio lateral, Vel-V: mean velocity-Vertical, Vel-AP: mean velocity-Antero Posterior, PPA: mean peak power acceleration, TA: mean tremor acceleration, TPA: mean total power acceleration, PPG: mean peak power gyroscope, TG: mean tremor gyroscope, TPG: mean total power gyroscope.

TABLE 10
Parameters extracted from the Vision Contrast test. NPL-ML: mean normalized path
length - mean medio lateral, NPL-V: mean normalized path length-Vertical, NPL-AP:
mean normalized path length-Antero Posterior, Vel-ML: mean velocity-Medio lateral,
Vel-V: mean velocity-Vertical, Vel-AP: mean velocity-Antero Posterior, PPA:
mean peak power acceleration, TA: mean tremor acceleration, TPA: mean
total power acceleration, PPG: mean peak power gyroscope, TG: mean
tremor gyroscope, TPG: mean total power gyroscope.

| | Score | Score | NPL- | NPL- | NPL- | Vel- | Vel- | Vel- | | | | | | |
|---|---|---|---|---|---|---|---|---|---|---|---|---|---|---|
| Rep. | left | right | ML | V | AP | ML | V | AP | PPA | TA | TPA | PPG | TG | TPG |
| 1 | 1.70 | 1.70 | 0.049 | 0.062 | 0.018 | 0.05 | 0.05 | 0.144 | 0.002 | 0.018 | 0.288 | 0.005 | 0.046 | 0.855 |
| 2 | 1.70 | 1.70 | 0.121 | 0.135 | 0.021 | 0.031 | 0.029 | 0.177 | 0.004 | 0.018 | 0.252 | 0.026 | 0.105 | 1.843 |
| 3 | 1.70 | 1.70 | 0.107 | 0.101 | 0.016 | 0.043 | 0.032 | 0.132 | 0.003 | 0.020 | 0.420 | 0.003 | 0.032 | 0.487 |
| 4 | 1.55 | 1.55 | 0.096 | 0.074 | 0.021 | 0.031 | 0.028 | 0.184 | 0.003 | 0.018 | 0.275 | 0.012 | 0.067 | 0.998 |
| 5 | 1.70 | 1.40 | 0.114 | 0.112 | 0.019 | 0.029 | 0.026 | 0.155 | 0.002 | 0.012 | 0.173 | 0.009 | 0.074 | 1.327 |

TABLE 11
Parameters extracted from the Catch a Cloud test. NPL-ML: mean normalized path
length - mean medio lateral, NPL-V: mean normalized path length-Vertical, NPL-AP:
mean normalized path length-Antero Posterior, Vel-ML: mean velocity-Medio lateral,
Vel-V: mean velocity-Vertical, Vel-AP: mean velocity-Antero Posterior, PPA:
mean peak power acceleration, TA: mean tremor acceleration, TPA: mean
total power acceleration, PPG: mean peak power gyroscope, TG: mean
tremor gyroscope, TPG: mean total power gyroscope.

| | | NPL- | NPL- | NPL- | Vel- | Vel- | Vel- | | | | | | |
|---|---|---|---|---|---|---|---|---|---|---|---|---|---|
| Rep. | Score | ML | V | AP | ML | V | AP | PPA | TA | TPA | PPG | TG | TPG |
| 1 | 30 | 0.160 | 0.012 | 0.047 | 0.824 | 0.039 | 0.436 | 0.002 | 0.011 | 0.103 | 0.019 | 0.074 | 0.145 |
| 2 | 30 | 0.210 | 0.019 | 0.051 | 0.847 | 0.018 | 0.429 | 0.004 | 0.019 | 0.202 | 0.017 | 0.077 | 0.212 |
| 3 | 32 | 0.173 | 0.014 | 0.047 | 0.322 | 0.037 | 0.419 | 0.003 | 0.014 | 0.125 | 0.009 | 0.040 | 0.183 |
| 4 | 31 | 0.173 | 0.014 | 0.048 | 0.677 | 0.017 | 0.398 | 0.003 | 0.013 | 0.125 | 0.016 | 0.065 | 0.228 |
| | 32 | | | | | | | | | | | | |
| 5 | 31 | 0.181 | 0.014 | 0.046 | 0.398 | 0.020 | 0.431 | 0.002 | 0.014 | 0.095 | 0.009 | 0.047 | 0.165 |
| 6 | 33 | 0.244 | 0.016 | 0.049 | 0.675 | 0.020 | 0.413 | 0.003 | 0.016 | 0.139 | 0.019 | 0.069 | 0.282 |
| 7 | 23 | 0.178 | 0.015 | 0.047 | 0.415 | 0.059 | 0.524 | 0.003 | 0.015 | 0.120 | 0.009 | 0.047 | 0.131 |
| 8 | 27 | 0.144 | 0.013 | 0.048 | 0.289 | 0.032 | 0.419 | 0.003 | 0.013 | 0.128 | 0.006 | 0.033 | 0.159 |
| 9 | 29 | 0.205 | 0.02 | 0.049 | 1.112 | 0.021 | 0.456 | 0.004 | 0.020 | 0.176 | 0.026 | 0.100 | 0.273 |
| 10 | 27 | 0.191 | 0.010 | 0.049 | 1.060 | 0.015 | 0.440 | 0.002 | 0.010 | 0.106 | 0.029 | 0.093 | 0.229 |

TABLE 12
Parameters extracted from the Screen to Nose test. NT: number of touches, TT:
mean touch time, TV: mean touch velocity, ST: mean stretch time, SV: mean
stretch velocity, TJ: mean touch jerk swayness, SJ: mean stretch jerk swayness,
TTA: mean touch tremor acceleration, TTG: mean touch tremor gyroscope, STA:
mean stretch tremor acceleration, STG: mean stretch tremor gyroscope.

| Rep. | NT | TT | TV | ST | SV | TJ | SJ | TTA | TTG | STA | STG |
|---|---|---|---|---|---|---|---|---|---|---|---|
| 1.1 | 10 | 0.456 | 0.657 | 1.1333 | 0.625 | 0.0635 | 0.084 | 0.142 | 1.023 | 0.108 | 0.846 |
| 1.2 | 9 | 0.536 | 0.662 | 1.0756 | 0.622 | 0.087 | 0.087 | 0.132 | 0.956 | 0.104 | 0.626 |
| 1.3 | 8 | 1.231 | 1.274 | 0.49 | 1.374 | 0.3316 | 0.3022 | 0.169 | 0.874 | 0.218 | 1.188 |
| 1.4 | 9 | 1.057 | 1.589 | 0.635 | 1.755 | 0.142 | 0.257 | 0.137 | 0.746 | 0.239 | 1.165 |
| 2.1 | 9 | 0.577 | 0.819 | 1.0911 | 0.680 | 0.058 | 0.073 | 0.125 | 0.840 | 0.091 | 0.702 |
| 2.2 | 8 | 0.546 | 0.889 | 1.135 | 0.770 | 0.0613 | 0.062 | 0.124 | 0.827 | 0.093 | 0.605 |
| 2.3 | 6 | 1.913 | 0.623 | 0.56 | 0.797 | 0.219 | 0.088 | 0.114 | 0.643 | 0.132 | 0.760 |
| 2.4 | 6 | 1.53 | 1.086 | 0.71 | 0.9752 | 0.079 | 0.053 | 0.077 | 0.403 | 0.115 | 0.706 |
| 3.1 | 7 | 0.934 | 0.572 | 1.22 | 0.501 | 0.060 | 0.084 | 0.113 | 0.700 | 0.100 | 0.653 |
| 3.2 | 6 | 0.786 | 0.483 | 1.507 | 0.448 | 0.064 | 0.064 | 0.094 | 0.621 | 0.081 | 0.596 |
| 3.3 | 7 | 1.494 | 1.054 | 0.491 | 1.242 | 0.324 | 0.1429 | 0.152 | 0.760 | 0.218 | 0.783 |
| 3.4 | 7 | 1.574 | 0.546 | 0.637 | 0.528 | 0.142 | 0.0894 | 0.111 | 0.565 | 0.157 | 0.734 |
| 4.1 | 9 | 0.542 | 0.5325 | 1.15 | 0.5957 | 0.043 | 0.0974 | 0.110 | 0.775 | 0.119 | 0.816 |
| 4.2 | 9 | 0.797 | 0.660 | 0.937 | 0.530 | 0.046 | 0.094 | 0.105 | 0.797 | 0.119 | 0.704 |
| 4.3 | 9 | 1.151 | 0.738 | 0.575 | 0.697 | 0.295 | 0.193 | 0.174 | 0.880 | 0.183 | 0.959 |
| 4.4 | 8 | 1.197 | 0.699 | 0.76 | 0.704 | 0.131 | 0.161 | 0.116 | 0.620 | 0.150 | 0.797 |
| 5.1 | 9 | 0.497 | 0.480 | 1.082 | 0.592 | 0.043 | 0.068 | 0.096 | 0.724 | 0.095 | 0.689 |
| 5.1 | 8 | 0.64 | 0.7979 | 1.03 | 0.687 | 0.047 | 0.047 | 0.087 | 0.608 | 0.082 | 0.472 |
| 5.3 | 8 | 1.167 | 0.659 | 0.547 | 0.718 | 0.1286 | 0.1441 | 0.117 | 0.701 | 0.183 | 1.028 |
| 5.4 | 8 | 1.21 | 1.131 | 0.612 | 1.068 | 0.071 | 0.125 | 0.093 | 0.562 | 0.177 | 0.782 |
| 6.1 | 9 | 0.5 | 0.547 | 1.285 | 0.514 | 0.032 | 0.043 | 0.104 | 0.639 | 0.069 | 0.530 |
| 6.2 | 7 | 0.571 | 0.8045 | 1.288 | 0.707 | 0.0389 | 0.051 | 0.086 | 0.575 | 0.071 | 0.498 |
| 6.3 | 7 | 1.38 | 0.761 | 0.554 | 0.800 | 0.141 | 0.09 | 0.104 | 0.576 | 0.157 | 0.713 |
| 6.4 | 8 | 1.362 | 0.785 | 0.6114 | 0.816 | 0.081 | 0.084 | 0.087 | 0.499 | 0.149 | 0.877 |
| 7.1 | 10 | 0.466 | 0.700 | 0.878 | 0.683 | 0.094 | 0.088 | 0.158 | 1.100 | 0.124 | 0.750 |
| 7.2 | 11 | 0.496 | 0.870 | 0.8418 | 0.719 | 0.118 | 0.097 | 0.176 | 1.054 | 0.138 | 0.729 |
| 7.3 | 10 | 0.944 | 0.709 | 0.447 | 0.814 | 0.220 | 0.260 | 0.167 | 0.881 | 0.248 | 1.087 |
| 7.4 | 10 | 0.967 | 1.31 | 0.542 | 1.393 | 0.211 | 0.296 | 0.158 | 0.705 | 0.255 | 1.237 |
| 8.1 | 9 | 0.973 | 0.325 | 0.693 | 0.185 | 0.036 | 0.023 | 0.064 | 0.428 | 0.066 | 0.354 |
| 8.2 | 5 | 1.452 | 0.4765 | 1.172 | 0.356 | 0.042 | 0.018 | 0.055 | 0.422 | 0.038 | 0.304 |
| 8.3 | 6 | 2.045 | 0.668 | 0.75 | 0.570 | 0.169 | 0.093 | 0.102 | 0.494 | 0.121 | 0.514 |
| 8.4 | 5 | 2.496 | 0.4185 | 0.708 | 0.39 | 0.098 | 0.073 | 0.07 | 0.43 | 0.09 | 0.471 |
| 9.1 | 8 | 0.84 | 0.742 | 1.035 | 0.570 | 0.062 | 0.045 | 0.111 | 0.821 | 0.075 | 0.535 |
| 9.2 | 7 | 0.74 | 0.588 | 1.24 | 0.581 | 0.050 | 0.039 | 0.100 | 0.703 | 0.076 | 0.500 |
| 9.3 | 7 | 1.6257 | 0.668 | 0.548 | 0.730 | 0.261 | 0.115 | 0.124 | 0.612 | 0.156 | 0.545 |
| 9.4 | 7 | 1.2914 | 0.777 | 0.737 | 0.858 | 0.086 | 0.093 | 0.092 | 0.448 | 0.132 | 0.659 |
| 10.1 | 9 | 0.400 | 0.877 | 1.306 | 0.893 | 0.058 | 0.114 | 0.139 | 1.009 | 0.103 | 0.779 |
| 10.2 | 8 | 0.5112 | 0.865 | 1.257 | 0.762 | 0.070 | 0.081 | 0.125 | 0.926 | 0.090 | 0.640 |
| 10.3 | 7 | 1.3571 | 0.885 | 0.582 | 0.933 | 0.148 | 0.115 | 0.117 | 0.634 | 0.177 | 1.001 |
| 10.4 | 9 | 1.2111 | 0.778 | 0.602 | 0.772 | 0.082 | 0.097 | 0.095 | 0.539 | 0.158 | 0.968 |

TABLE 13
Parameters extracted from the M-SDMT test. NPL-ML: mean normalized path length - mean
medio lateral, NPL-V: mean normalized path length-Vertical, NPL-AP: mean normalized
path length-Antero Posterior, Vel-ML: mean velocity-Medio lateral, Vel-V: mean velocity-
Vertical, Vel-AP: mean velocity-Antero Posterior, PPA: mean peak power acceleration,
TA: mean tremor acceleration, TPA: mean total power acceleration, PPG: mean peak power
gyroscope, TG: mean tremor gyroscope, TPG: mean total power gyroscope.

| | NPL- | NPL- | NPL- | Vel- | Vel- | Vel- | | | | | | |
|---|---|---|---|---|---|---|---|---|---|---|---|---|
| Rep. | ML | V | AP | ML | V | AP | PPA | TA | TPA | PPG | TG | TPG |
| 1 | 0.111 | 0.0596 | 0.011 | 0.042 | 0.091 | 0.122 | 0.001 | 0.013 | 0.367 | 0.006 | 0.111 | 2.110 |
| 2 | 0.108 | 0.104 | 0.012 | 0.020 | 0.040 | 0.116 | 0.002 | 0.02 | 0.507 | 0.007 | 0.108 | 1.625 |
| 3 | 0.099 | 0.0786 | 0.012 | 0.018 | 0.0431 | 0.1223 | 0.002 | 0.013 | 0.288 | 0.004 | 0.099 | 0.886 |
| 4 | 0.090 | 0.091 | 0.014 | 0.024 | 0.0592 | 0.1145 | 0.001 | 0.015 | 0.32 | 0.004 | 0.090 | 0.792 |
| 5 | 0.075 | 0.082 | 0.016 | 0.040 | 0.085 | 0.1515 | 0.002 | 0.015 | 0.389 | 0.005 | 0.07 | 0.816 |

TABLE 14
Parameters extracted from theTightRope test.

| Rep. | NPL-ML | NPL-AP | JerkSw |
|---|---|---|---|
| 1.1 | 0.324 | 0.134 | 0.0116 |
| 1.2 | 0.268 | 0.132 | 0.008 |
| 2.1 | 0.232 | 0.134 | 0.006 |
| 2.2 | 0.229 | 0.132 | 0.001 |
| 3.1 | 0.239 | 0.134 | 0.005 |
| 3.2 | 0.262 | 0.132 | 0.003 |
| 4.1 | 0.270 | 0.133 | 0.002 |
| 4.2 | 0.286 | 0.132 | 0.002 |
| 5.1 | 0.358 | 0.133 | 0.003 |
| 5.2 | 0.223 | 0.132 | 0.002 |
| 6.1 | 0.267 | 0.132 | 0.004 |
| 6.2 | 0.265 | 0.132 | 0.005 |
| 7.1 | 0.252 | 0.133 | 0.004 |
| 7.2 | 0.224 | 0.132 | 0.002 |
| 8.1 | 0.413 | 0.134 | 0.008 |
| 8.2 | 0.501 | 0.132 | 0.003 |
| 9.1 | 0.245 | 0.133 | 0.005 |
| 9.2 | 0.281 | 0.133 | 0.002 |
| 10.1 | 0.258 | 0.132 | 0.005 |
| 10.2 | 0.210 | 0.132 | 0.003 |

NPL-ML: mean normalized path length-Medio lateral, NPL-AP: mean normalized path length-Antero Posterior, JerkSw: mean jerk swayness.

TABLE 15
Parameters extracted from the Wobbler test. NPL-ML: mean normalized path length - mean
medio lateral, NPL-V: mean normalized path length-Vertical, NPL-AP: mean normalized
path length-Antero Posterior, Vel-ML: mean velocity-Medio lateral, Vel-V: mean velocity-
Vertical, Vel-AP: mean velocity-Antero Posterior, PPA: mean peak power acceleration,
TA: mean tremor acceleration, TPA: mean total power acceleration, PPG: mean peak power
gyroscope, TG: mean tremor gyroscope, TPG: mean total power gyroscope.

| | NPL- | NPL- | NPL- | Vel- | Vel- | Vel- | | | | | | |
|---|---|---|---|---|---|---|---|---|---|---|---|---|
| Rep. | ML | V | AP | ML | V | AP | PPA | TA | TPA | PPG | TG | TPG |
| 1.1 | 0.238 | 0.2056 | 0.133 | 0.071 | 0.123 | 1.1634 | 0.005 | 0.020 | 0.097 | 0.041 | 0.120 | 0.763 |
| 1.2 | 0.233 | 0.220 | 0.131 | 0.064 | 0.080 | 1.211 | 0.010 | 0.024 | 0.147 | 0.126 | 0.217 | 1.518 |
| 1.3 | 0.273 | 0.195 | 0.131 | 0.023 | 0.062 | 1.288 | 0.005 | 0.015 | 0.068 | 0.006 | 0.031 | 0.114 |
| 1.4 | 0.311 | 0.219 | 0.131 | 0.026 | 0.090 | 1.196 | 0.008 | 0.025 | 0.126 | 0.031 | 0.127 | 0.451 |
| 2.1 | 0.316 | 0.526 | 0.136 | 0.039 | 0.018 | 1.121 | 0.011 | 0.024 | 0.129 | 0.022 | 0.090 | 0.435 |
| 2.2 | 0.233 | 0.288 | 0.133 | 0.036 | 0.041 | 1.138 | 0.005 | 0.014 | 0.076 | 0.013 | 0.056 | 0.204 |
| 2.3 | 0.241 | 0.365 | 0.130 | 0.107 | 0.038 | 1.34 | 0.006 | 0.02 | 0.097 | 0.028 | 0.077 | 0.310 |
| 2.4 | 0.242 | 0.272 | 0.132 | 0.045 | 0.017 | 1.172 | 0.003 | 0.010 | 0.056 | 0.007 | 0.025 | 0.125 |
| 3.1 | 0.237 | 0.214 | 0.129 | 0.047 | 0.08 | 1.306 | 0.007 | 0.015 | 0.078 | 0.018 | 0.059 | 0.325 |
| 3.2 | 0.263 | 0.314 | 0.132 | 0.034 | 0.024 | 1.185 | 0.004 | 0.013 | 0.066 | 0.008 | 0.027 | 0.147 |
| 3.3 | 0.195 | 0.297 | 0.131 | 0.073 | 0.020 | 1.259 | 0.005 | 0.01 | 0.053 | 0.017 | 0.046 | 0.261 |
| 3.4 | 0.217 | 0.244 | 0.132 | 0.037 | 0.051 | 1.2022 | 0.005 | 0.014 | 0.066 | 0.017 | 0.048 | 0.255 |
| 4.1 | 0.278 | 0.235 | 0.134 | 0.075 | 0.131 | 1.143 | 0.004 | 0.019 | 0.077 | 0.031 | 0.096 | 0.496 |
| 4.2 | 0.226 | 0.506 | 0.131 | 0.041 | 0.022 | 1.241 | 0.008 | 0.019 | 0.108 | 0.020 | 0.072 | 0.385 |
| 4.3 | 0.242 | 0.188 | 0.132 | 0.053 | 0.075 | 1.253 | 0.006 | 0.019 | 0.083 | 0.017 | 0.061 | 0.238 |
| 4.4 | 0.225 | 0.251 | 0.130 | 0.088 | 0.031 | 1.256 | 0.004 | 0.015 | 0.069 | 0.018 | 0.063 | 0.239 |
| 5.1 | 0.263 | 0.620 | 0.133 | 0.043 | 0.0134 | 1.290 | 0.008 | 0.019 | 0.126 | 0.008 | 0.038 | 0.211 |
| 5.2 | 0.207 | 0.216 | 0.132 | 0.053 | 0.043 | 1.183 | 0.004 | 0.012 | 0.060 | 0.026 | 0.058 | 0.370 |
| 5.3 | 0.314 | 0.315 | 0.131 | 0.026 | 0.018 | 1.263 | 0.006 | 0.016 | 0.076 | 0.019 | 0.065 | 0.341 |
| 5.4 | 0.224 | 0.235 | 0.1314 | 0.057 | 0.050 | 1.189 | 0.005 | 0.013 | 0.060 | 0.012 | 0.048 | 0.198 |
| 6.1 | 0.254 | 0.206 | 0.1333 | 0.037 | 0.099 | 1.194 | 0.006 | 0.023 | 0.125 | 0.034 | 0.105 | 0.556 |
| 6.2 | 0.285 | 0.253 | 0.132 | 0.031 | 0.0724 | 1.171 | 0.010 | 0.022 | 0.141 | 0.024 | 0.079 | 0.409 |
| 6.3 | 0.219 | 0.192 | 0.131 | 0.078 | 0.046 | 1.272 | 0.004 | 0.012 | 0.071 | 0.032 | 0.076 | 0.447 |
| 6.4 | 0.243 | 0.229 | 0.1303 | 0.032 | 0.016 | 1.2226 | 0.005 | 0.015 | 0.070 | 0.014 | 0.057 | 0.243 |
| 7.1 | 0.241 | 0.358 | 0.129 | 0.051 | 0.0212 | 1.2736 | 0.006 | 0.021 | 0.093 | 0.033 | 0.088 | 0.529 |
| 7.2 | 0.269 | 0.642 | 0.132 | 0.093 | 0.023 | 1.2204 | 0.006 | 0.019 | 0.110 | 0.020 | 0.076 | 0.447 |
| 7.3 | 0.204 | 0.199 | 0.1334 | 0.115 | 0.143 | 1.188 | 0.005 | 0.016 | 0.086 | 0.020 | 0.059 | 0.301 |
| 7.4 | 0.240 | 0.320 | 0.129 | 0.072 | 0.023 | 1.263 | 0.015 | 0.025 | 0.146 | 0.061 | 0.099 | 0.568 |
| 8-1 | 0.268 | 0.254 | 0.133 | 0.04 | 0.044 | 1.096 | 0.003 | 0.016 | 0.070 | 0.009 | 0.034 | 0.144 |
| 8.2 | 0.392 | 0.299 | 0.131 | 0.035 | 0.025 | 1.217 | 0.023 | 0.028 | 0.104 | 0.046 | 0.055 | 0.202 |
| 8.3 | 0.272 | 0.412 | 0.133 | 0.039 | 0.022 | 1.159 | 0.003 | 0.010 | 0.045 | 0.014 | 0.051 | 0.280 |
| 8.4 | 0.290 | 0.33 | 0.132 | 0.066 | 0.028 | 1.175 | 0.005 | 0.015 | 0.069 | 0.009 | 0.041 | 0.188 |
| 9.1 | 0.201 | 0.215 | 0.132 | 0.084 | 0.055 | 1.238 | 0.005 | 0.014 | 0.079 | 0.029 | 0.078 | 0.43 |
| 9.2 | 0.265 | 0.280 | 0.1309 | 0.062 | 0.042 | 1.216 | 0.004 | 0.014 | 0.088 | 0.013 | 0.056 | 0.326 |
| 9.3 | 0.207 | 0.486 | 0.133 | 0.188 | 0.019 | 1.222 | 0.006 | 0.025 | 0.116 | 0.028 | 0.102 | 0.481 |
| 9.4 | 0.244 | 0.234 | 0.131 | 0.073 | 0.0515 | 1.174 | 0.002 | 0.013 | 0.054 | 0.006 | 0.023 | 0.113 |
| 10.1 | 0.193 | 0.224 | 0.134 | 0.170 | 0.049 | 1.123 | 0.007 | 0.021 | 0.109 | 0.022 | 0.076 | 0.384 |
| 10.2 | 0.299 | 0.207 | 0.131 | 0.046 | 0.038 | 1.226 | 0.016 | 0.025 | 0.127 | 0.034 | 0.058 | 0.303 |
| 10.3 | 0.189 | 0.214 | 0.135 | 0.171 | 0.034 | 1.1021 | 0.007 | 0.024 | 0.114 | 0.031 | 0.101 | 0.523 |
| 10.4 | 0.210 | 0.279 | 0.131 | 0.040 | 0.024 | 1.190 | 0.009 | 0.022 | 0.126 | 0.027 | 0.072 | 0.362 |

TABLE 16
Parameters extracted from the Musical Chairs test. NS: number
of squats, TS: mean time to sit, mean velocity to sit, JS:
mean jerk swayness to sit, TSt: mean time to stand, VSt:
mean velocity to stand, JSt: mean jerk swayness to stand.

| Rep. | NS | TS | VS | JS | TSt | VSt | JSt |
|---|---|---|---|---|---|---|---|
| 1 | 8 | 1.161 | 0.337 | 0.049 | 2.794 | 0.32 | 0.215 |
| 2 | 8 | 1.91 | 0.275 | 0.088 | 1.488 | 0.268 | 0.122 |
| 3 | 10 | 1.174 | 0.428 | 0.072 | 2.615 | 0.421 | 0.138 |
| 4 | 14 | 0.94 | 0.295 | 0.037 | 1.126 | 0.268 | 0.074 |
| 5 | 8 | 2.201 | 0.412 | 0.098 | 1.91 | 0.336 | 0.091 |
| 6 | 9 | 1.580 | 0.203 | 0.106 | 1.795 | 0.293 | 0.081 |
| 7 | 3 | 0.906 | 3.790 | 0.101 | 9.16 | 2.385 | 1.068 |
| 8 | 12 | 0.955 | 0.392 | 0.091 | 1.128 | 0.271 | 0.052 |
| 9 | 10 | 1.54 | 0.261 | 0.077 | 1.07 | 0.273 | 0.054 |
| 10 | 8 | 1.1 | 0.311 | 0.046 | 2.773 | 0.289 | 0.132 |

TABLE 17
Parameters extracted from the U-turns test.

| Rep. | NU | TU | VU |
|---|---|---|---|
| 1 | 5 | 1.46 | 144.390 |
| 2 | 7 | 1.094 | 164.534 |
| 3 | 7 | 1.303 | 160.996 |
| 4 | 8 | 1.082 | 166.316 |
| 5 | 8 | 1.09 | 165.179 |
| 6 | 7 | 1.085 | 165.817 |
| 7 | 8 | 1.065 | 169.054 |
| 8 | 8 | 1.082 | 166.4 |
| 9 | 8 | 1.102 | 163.311 |
| 10 | 7 | 1.085 | 165.8791 |

NU: number of u-turns, TU: mean time of u-turns, VU: mean velocity of u-turns.

TABLE 18
Parameters extracted from the Climbing Stairs test. NS: number of steps, C:
mean cadence, ST: mean step time, SR: mean step regularity, SDTW: mean step
dynamic time warping, SJ: mean step jerk swayness, Str: number of strides,
StrT: mean stride time, StrR: mean stride regularity, StrDTW: mean stride
dynamic time warping, StrJ: mean stride jerk swayness, Gs: gait Symmetric.

| Rep. | NS | Ca | ST | SR | SDTW | SJ | Str | StrT | StrR | StrDTW | StrJ | Gs |
|---|---|---|---|---|---|---|---|---|---|---|---|---|
| 1 | 32 | 1.976 | 0.506 | 0.539 | 0.704 | 0.039 | 64 | 1.012 | 0.378 | 0.696 | 0.079 | 0.701 |
| 2 | 36 | 1.076 | 0.538 | 0.499 | 0.676 | 0.033 | 72 | 1.076 | 0.328 | 0.669 | 0.070 | 0.657 |
| 3 | 36 | 1.022 | 0.511 | 0.584 | 0.700 | 0.038 | 72 | 1.022 | 0.493 | 0.706 | 0.079 | 0.844 |
| 4 | 35 | 0.904 | 0.452 | 0.609 | 0.695 | 0.032 | 70 | 904 | 0.530 | 0.674 | 0.064 | 0.,870 |
| 5 | 41 | 1.082 | 0.541 | 0.504 | 0.670 | 0.019 | 82 | 1.082 | 0.469 | 0.684 | 0.041 | 0.930 |
| 6 | 45 | 0.984 | 0.492 | 0.454 | 0.665 | 0.026 | 90 | 984 | 0.265 | 0.640 | 0.050 | 0.583 |
| 7 | 37 | 1.054 | 0.527 | 0.430 | 0.641 | 0.023 | 74 | 1.054 | 0.345 | 0.662 | 0.052 | 0.802 |
| 8 | 37 | 1.006 | 0.503 | 0.494 | 0.677 | 0.024 | 74 | 1.006 | 0.437 | 0.660 | 0.056 | 0.884 |
| 9 | 42 | 1.19 | 0.595 | 0.530 | 0.678 | 0.020 | 84 | 1.190 | 0.302 | 0.691 | 0.096 | 0.569 |
| 10 | 44 | 1.136 | 0.568 | 0.434 | 0.650 | 0.034 | 88 | 1.136 | 0.354 | 0.650 | 0.063 | 0.815 |

TABLE 19
Parameters extracted from the Two-min Walk test. NS: number of steps, C: mean cadence, ST: mean
step time, SR: mean step regularity, SDTW: mean step dynamic time warping, SJ: mean step jerk
swayness, Str: number of strides, StrT: mean stride time, StrR: mean stride regularity, StrDTW:
mean stride dynamic time warping, StrJ: mean stride jerk swayness, Gs: gait Symmetric.

| Rep | NS | Ca | ST | SR | SDTW | SJ | Str | StrT | StrR | StrDTW | StrJ | Gs |
|---|---|---|---|---|---|---|---|---|---|---|---|---|
| 1 | 72 | 0.816 | 1.225 | 0.389 | 0.716 | 0.1735 | 144 | 2.450 | 0.301 | 0.651 | 0.436 | 0.774 |
| 2 | 120 | 1.515 | 0.660 | 0.469 | 0.697 | 0.0802 | 240 | 1.320 | 0.287 | 0.667 | 0.221 | 0.612 |
| 3 | 82 | 0.740 | 1.352 | 0.471 | 0.762 | 0.168 | 164 | 2.704 | 0.403 | 0.675 | 0.366 | 0.856 |
| 4 | 150 | 1.828 | 0.547 | 0.728 | 0.777 | 0.060 | 300 | 1.094 | 0.526 | 0.692 | 0.146 | 0.723 |
| 5 | 68 | 0.714 | 1.400 | 0.427 | 0.749 | 0.249 | 136 | 2.800 | 0.338 | 0.676 | 0.533 | 0.792 |
| 6 | 86 | 0.930 | 1.075 | 0.480 | 0.710 | 0.088 | 172 | 2.150 | 0.300 | 0.658 | 0.285 | 0.625 |
| 7 | 72 | 0.884 | 1.131 | 0.436 | 0.734 | 0.166 | 144 | 2.262 | 0.340 | 0.647 | 0.398 | 0.780 |
| 8 | 60 | 0.735 | 1.36 | 0.343 | 0.733 | 0.218 | 120 | 2.720 | 0.392 | 0.555 | 0.48 | 0.875 |
| 9 | 73 | 0.822 | 1.217 | 0.333 | 0.705 | 0.225 | 146 | 2.434 | 0.282 | 0.634 | 0.501 | 0.847 |
| 10 | 72 | 0.881 | 1.135 | 0.422 | 0.731 | 0.165 | 144 | 2.270 | 0.274 | 0.651 | 0.386 | 0.649 |

1. A method for providing a state of a disease in a subject, the method comprising the steps of:
a) determining at least two parameters indicative of a disease state in the subject at a first time point;
b) combining the determined parameters in a) to provide a signature indicative of the disease state at the first time point;
c) repeating steps a) and b) to provide at least one further signature at a second time point;
d) combining the provided signatures in step c) to provide a progression marker indicative of the disease state in the subject.

2. The method of claim 1, further comprising step e) repeating steps a) to d) in order to monitor disease progression in the subject based on an alteration of a final signature provided in step d).

3. The method of claim 1, wherein the disease is a disease of the central nervous system (CNS).

4. The method of claim 1, wherein the at least two parameters are provided based on data obtained from:
imaging techniques;
(ii) patient surveys regarding symptoms experienced by the subject;
(iii) environment data including weather information, vision tests, social interaction assessment, quality of life;
(iv) cognitive tests;
(v) physical tests; and/or
(vi) biochemical marker determination.

5. The method of claim 4, wherein
the cognitive tests comprise eSDMT, language testing, problem solving testing, memory testing, focus testing, mood testing and/or mental agility testing; and/or
the physical tests comprise walking, tight rope, climbing stairs, wobbler, U-turn, musical chairs, figures writing, screen to nose, cuddle a cloud, standing-up/sitting-down, level of activity, sleep, and/or heart rate.

6. The method of claim 4, wherein data results from passive data collection and/or wherein data results from active data collection.

7. The method of claim 1, wherein at least one parameter is determined by or using a mobile device.

8. The method of claim 2, wherein the respective method steps are repeated according to steps c) or e), respectively, after a time interval determined based on the disease state.

9. The method of claim 2, the method further comprising a step of selecting the parameters to be determined in step a) based on the disease state and/or disease progression.

10. The method of claim 1, wherein in steps b) and/or d), the parameters and/or signatures are combined in a weighted manner.

11. The method of claim 1, wherein the method comprises using statistical methods, pattern recognition techniques, digital image processing, and/or artificial intelligence techniques.

12. A method for determining efficacy of therapy of a disease, the method comprising using the method of claim 2, wherein therapy is determined to be efficient if the alteration of the final signature provided in step d) is below a pre-determined threshold.

13. A method of treating a subject with a disease of the central nervous system (CNS), wherein treatment is initiated/adapted based on the disease state and/or progression of the disease determined by the method of claim 1, wherein treatment administered to the subject comprises interferon beta-1a, interferon beta-1b, an agent specifically binding to CD52, an agent specifically-binding to CD20, an agent specifically binding to integrin.

14. A mobile device comprising a processor, at least one sensor, a database and software which is tangibly embedded in said device and, when running on said device, carries out the method of claim 1.

15. A system comprising a mobile device comprising at least one sensor and a remote device comprising a processor and a database as well as software which is tangibly embedded to said device and, when running on said device, carries out the method of claim 1, wherein said mobile device and said remote device are operatively linked to each other.

16. The method of claim 3, wherein the disease of the CNS is multiple sclerosis (MS), progressing MS, relapsing-remitting MS with clinical disease activity, relapsing-remitting MS with disability progression, secondary progressive MS, secondary progressive MS with disability progression, primary progressive MS, or primary progressive MS with disability progression

17. The method of claim 3, wherein the imaging techniques includes magnetic resonance imaging (MRI) and/or optical coherence tomography (OCT);
wherein the physical tests comprises testing motoric and/or fine motoric capabilities and/or function, walking, vision, sleep; and
wherein the biochemical marker determination comprises determining a biochemical marker from a sample obtained from the subject, wherein the sample comprises blood, spinal cord fluid, cerebral spinal fluid, saliva and/or lymph.

18. The method of claim 7, wherein said mobile device comprises a smartphone, smartwatch, wearable sensor, portable multimedia device or tablet computer.

19. The method of claim 11, wherein the artificial intelligence techniques includes machine learning and/or neural networks, wherein the used method/technique is adapted based on the provided signatures.

20. The pharmaceutical composition of claim 13, wherein the pharmaceutical composition comprises glatiramer, teriflunomide, fingolimod, dimethyl fumarate, siponimod, cladribine, alemtuzumab, mitoxantrone, ocrelizumab and/or natalizumab.
